SEQUENCE LISTING (24 sequences)

SEQ ID NO: 1 - protein, 15 aa, synthetic construct
TLLVDVEPKVKSKKR

SEQ ID NO: 2 - protein, 119 aa, Homo sapiens
MKLVRFLMKLSHETVTIELKNGTQVHGTITGVDVSMNTHLKAVKMTLKNREPVQLETLSIRGNNIRYFILPDSLPLDTLLVDVEPKVKSKKREAVAGRGRGRGRGRGRGRGRGRGGPRR

SEQ ID NO: 3 - protein, 15 aa, synthetic construct
DYRMRCILQDGRIFI

SEQ ID NO: 4 - protein, 233 aa, Homo sapiens
KMLQHIDYRMRCILQDGRIFIGTFKAFDKHMNLILCDCDEFRKIKPKNSKQAEREEKRVLGLVLLRGENLVSMTVEGPPPKDTGIARVPLAGAAGGPGIGRAAGRGIPAGVPMPQAPAGLAGPVRGVGGPSQQVMTPQGRGTVAAAAAAATASIAGAPTQYPPGRGGPPPPMGRGAPPPGMMGPPPGMRPPMGPPMGIPPGRGTPMGMPPPGMRPPPPGMRGPPPPGMRPPRP

SEQ ID NO: 5 - protein, 15 aa, synthetic construct
LLKYLEAVEKVKRTR

SEQ ID NO: 6 - protein, 15 aa, synthetic construct
KRFLLAVDVSASMNQ

SEQ ID NO: 7 - protein, 538 aa, Homo sapiens
MEESVNQMQPLNEKQIANSQDGYVWQVTDMNRLHRFLCFGSEGGTYYIKEQKLGLENAEALIRLIEDGRGCEVIQEIKSFSQEGRTTKQEPMLFALAICSQCSDISTKQAAFKAVSEVCRIPTHLFTFIQFKKDLKESMKCGMWGRALRKAIADWYNEKGGMALALAVTKYKQRNGWSHKDLLRLSHLKPSSEGLAIVTKYITKGWKEVHELYKEKALSVETEKLLKYLEAVEKVKRTRDELEVIHLIEEHRLVREHLLTNHLKSKEVWKALLQEMPLTALLRNLGKMTANSVLEPGNSEVSLVCEKLCNEKLLKKARIHPFHILIALETYKTGHGLRGKLKWRPDEEILKALDAAFYKTFKTVEPTGKRFLLAVDVSASMNQRVLGSILNASTVAAAMCMVVTRTEKDSYVVAFSDEMVPCPVTTDMTLQQVLMAMSQIPAGGTDCSLPMIWAQKTNTPADVFIVFTDNETFAGGVHPAIALREYRKKMDIPAKLIVCGMTSNGFTIADPDDRGMLDMCGFDTGALDVIRNFTLDMI

SEQ ID NO: 8 - protein, 15 aa, synthetic construct
MVQIITYRDYLPLVL

SEQ ID NO: 9 - protein, 15 aa, synthetic construct
PRDFVNCSTLPALNL

SEQ ID NO: 10 - protein, 745 aa, Homo sapiens
MGVPFFSSLRCMVDLGPCWAGGLTAEMKLLLALAGLLAILATPQPSEGAAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 11 - protein, 6 aa, synthetic construct
NSASQS

SEQ ID NO: 12 - protein, 6 aa, synthetic construct
VYSSGN

SEQ ID NO: 13 - protein, 13 aa, synthetic construct
CVVPPGGGFKTIF

SEQ ID NO: 14 - protein, 5 aa, synthetic construct
MNHEY

SEQ ID NO: 15 - protein, 6 aa, synthetic construct
SMNVEV

SEQ ID NO: 16 - protein, 14 aa, synthetic construct
CASRTRTASYGYTF

SEQ ID NO: 17 - DNA, 18 bp, synthetic construct
  source 1..18: mol_type = other DNA
aacagtgcttctcagtct

SEQ ID NO: 18 - DNA, 18 bp, synthetic construct
  source 1..18: mol_type = other DNA
gtatactccagtggtaat

SEQ ID NO: 19 - DNA, 39 bp, synthetic construct
  source 1..39: mol_type = other DNA
tgtgtggtgcctcctgggggaggcttcaaaactatcttt

SEQ ID NO: 20 - DNA, 15 bp, synthetic construct
  source 1..15: mol_type = other DNA
atgaaccatgagtat

SEQ ID NO: 21 - DNA, 18 bp, synthetic construct
  source 1..18: mol_type = other DNA
tcaatgaatgttgaggtg

SEQ ID NO: 22 - DNA, 42 bp, synthetic construct
  source 1..42: mol_type = other DNA
tgtgccagcaggaccaggacagccagttatggctacaccttc

SEQ ID NO: 23 - protein, 209 aa, synthetic construct
MMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVPPGGGFKTIFGAGTRLFVKANIQNPDPAVYQLRDSKSSDKSVCLFTDFDSQTNVSQSKDSDVYITDKTVLDMRSMDFKSNSAVAWSNKSDFACANAF

SEQ ID NO: 24 - protein, 208 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRTRTASYGYTFGSGTRLTVVEDLNKVFPPEVAVFEPSEAEISHTQKATLVCLATGFFPDHVELSWWVNGKEVHSGVSTDPQPLKEQPALNDSRYCL

(19) United States
(12) Patent Application Publication
(10) Pub. No.: US 2024/0382588 A1
(43) Pub. Date: Nov. 21, 2024

(54) METHODS OF TREATMENT

(71) Applicant: MONASH UNIVERSITY, Clayton, Victoria (AU)

(72) Inventors: Joshua Daniel OOI, Melbourne (AU); Peter James EGGENHUIZEN, Melbourne (AU); Eric MORAND, Melbourne (AU)

(21) Appl. No.: 18/693,109

(22) PCT Filed: Sep. 21, 2022
(86) PCT No.: PCT/AU2022/051136

§ 371 Date: Mar. 18, 2024

(30) Foreign Application Priority Data
Sep. 21, 2021 (AU) 2021903030

(51) Int. Cl. A61K 39/00; A61P 37/06; C07K 14/725; C12N 5/0783

(52) U.S. Cl. CPC A61K 39/4611; A61K 39/4621; A61K 39/4632; A61K 39/46433; A61P 37/06; C07K 14/7051; C12N 5/0637; C12N 2510/00

(57) ABSTRACT

The present invention relates to methods and compositions for treating autoimmune or inflammatory disease characterised by an aberrant or inappropriate immune response to one or more of Ro60 protein; MPO protein; and Smith protein.

### FIELD OF THE INVENTION

The present invention relates to compositions and methods for the treatment of autoimmune or inflammatory diseases, particularly autoimmune diseases characterised by inappropriate or aberrant immune responses to one or more of Smith protein, Ro60 protein and MPO protein.

### RELATED APPLICATION

This application claims priority from Australian provisional application AU 2021903030, the entire contents of which are hereby incorporated by reference.

### BACKGROUND OF THE INVENTION

Autoimmune diseases arise from an aberrant immune response to healthy cells, tissues and organs. More than 80 autoimmune diseases are recognized in humans and collectively, these diseases affect more than 24 million individuals in the US alone.

In some instances, an individual may have more than one autoimmune disease, and auto-antibodies to a single “self-antigen” may be associated with more than one condition. Alternatively, individuals may present with multiple autoimmune diseases, for which there is no apparent common auto-antigen or cause of autoimmunity.

Despite significant research into autoimmune diseases, effective targeted therapies are lacking. Present treatments such as corticosteroids, methotrexate, hydroxycholorquine, other immunosuppressants (e.g., ciclosporin, leflunamide, azathioprine, to name a few), and non-steroidal anti-inflammatory drugs, non-specifically inhibit activation of the immune system rather than precisely inhibiting the specific autoimmunity associated with the disorder.

While many patients fail to respond or respond only partially to the standard of care medications listed above, the long-term use of high doses of corticosteroids and cytotoxic therapies may have profound side effects such as bone marrow depression, increased infections with opportunistic organisms, irreversible ovarian failure, alopecia and increased risk of malignancy. Infectious complications coincident with active autoimmune disease and its treatment with immunosuppressive medications are among the most common cause of death in patients with autoimmune disease.

There is a need for new or improved treatments for autoimmune disease, particularly treatments that target the disease-associated auto-antigens.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention is based on the surprising finding by the inventors, of a promiscuous self-epitope specific T cell receptor (TCR), capable of binding to multiple self-epitopes, and useful for treating various autoimmune diseases.

Accordingly, in a first aspect, the present invention provides a method of treating an autoimmune or inflammatory disease, the method comprising:
administering to a subject in need thereof, a population of regulatory T (T reg) cells expressing a binding protein on their surface,
wherein the binding protein comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto; and
wherein the Vβ domain comprises a CDR3 having an amino acid sequence as set forth in SEQ ID NO: 16, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto;
thereby treating the autoimmune disease or inflammatory disease,
wherein the autoimmune or inflammatory disease is characterised by an aberrant or inappropriate immune response to: Ro60 protein; myeloperoxidase (MPO) protein; Ro60 protein and MPO protein; Smith protein and Ro60 protein; Smith protein and MPO protein; or Smith protein, Ro60 protein and MPO protein.

In an embodiment of the first aspect of the invention there is also provided a use of a population of T reg cells as described above in the manufacture of a medicament for treating an autoimmune disease or inflammatory disease, preferably wherein the autoimmune or inflammatory disease is characterised by an aberrant or inappropriate immune response to: Ro60 protein; MPO protein; Ro60 protein and MPO protein; Smith protein and Ro60 protein; Smith protein and MPO protein; or Smith protein, Ro60 protein and MPO protein.

In a further embodiment of the first aspect of the invention there is provided a population of T reg cells as described above for use in the treatment of an autoimmune disease or inflammatory disease, preferably wherein the autoimmune or inflammatory disease is characterised by an aberrant or inappropriate immune response to: Ro60protein; MPO protein; Ro60 protein and MPO protein; Smith protein and Ro60 protein; Smith protein and MPO protein; or Smith protein, Ro60 protein and MPO protein.

In any embodiment, the autoimmune or inflammatory disease may be characterised by an aberrant or inappropriate immune response to Smith protein.

In any embodiment, the autoimmune or inflammatory disease may be characterised by an aberrant or inappropriate immune response to Ro60 protein.

In any embodiment, the autoimmune or inflammatory disease may be characterised by an aberrant or inappropriate immune response to Smith protein and to Ro60 protein.

In any embodiment, the autoimmune or inflammatory disease may be characterised by an aberrant or inappropriate immune response to MPO protein.

In any embodiment, the autoimmune or inflammatory disease may be characterised by an aberrant or inappropriate immune response to Ro60 protein and to MPO protein.

In any embodiment, the autoimmune or inflammatory disease may be characterised by an aberrant or inappropriate immune response to Smith protein, to Ro60 protein and to MPO protein.

In any embodiment, the aberrant or inappropriate immune response to Smith protein, to Ro60 protein and/or to MPO protein, comprises the formation of auto-antibodies to Smith protein, to Ro60 protein and/or to MPO protein.

Auto-antibodies to MPO protein may also be referred to herein as antineutrophil cytoplasmic antibodies (ANCAs), specifically, anti-MPO ANCAs.

In any embodiment, the autoimmune or inflammatory disease is one or more selected from the group consisting of: systemic lupus erythematosus (SLE), lupus nephritis, Sjogren's Syndrome, systemic sclerosis, inflammatory myositis, inflammatory rheumatism, autoimmune vasculitis, and microscopic polyangiitis. Preferably, the autoimmune or inflammatory disease may be SLE or lupus nephritis. Alternatively, the autoimmune or inflammatory disease may be Sjogren's Syndrome, preferably primary Sjogren's Syndrome. Further still, the autoimmune or inflammatory disease may be microscopic polyangiitis (MPA).

In any embodiment, the subject requiring treatment may require treatment for SLE and Sjogren's Syndrome, or for SLE and MPA, or for Sjogren's Syndrome and MPA, or for all three of SLE, Sjogren's Syndrome and MPA.

In a second aspect, there is provided a method of treating systemic lupus erythematosus (SLE) or lupus nephritis in a subject, the method comprising:
administering to a subject in need thereof, a population of T reg cells expressing a binding protein on their surface,
wherein the binding protein comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto; and
wherein the Vβ domain comprises a CDR3 having an amino acid sequence as set forth in SEQ ID NO: 16, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto;
thereby treating the SLE or lupus nephritis in the subject,
wherein the SLE or lupus nephritis is characterised by an aberrant or inappropriate immune response to: Ro60 protein; or Smith protein and Ro60 protein. Preferably, the method also comprises treating Sjogren's Syndrome and/or MPA in the subject.

In an embodiment of the second aspect of the invention there is also provided a use of a population of T reg cells as described above in the manufacture of a medicament for treating SLE or lupus nephritis in a subject, preferably wherein the SLE or lupus nephritis is characterised by an aberrant or inappropriate immune response to: Ro60 protein; or Smith protein and Ro60 protein. Preferably, the medicament is also for treating Sjogren's Syndrome and/or MPA in the subject.

In a further embodiment of the second aspect of the invention there is provided a population of T reg cells as described above for use in the treatment of SLE or lupus nephritis, preferably wherein the SLE or lupus nephritis is characterised by an aberrant or inappropriate immune response to: Ro60 protein; or Smith protein and Ro60 protein. Preferably, the use also comprises treating Sjogren's Syndrome and/or MPA in the subject.

In still a third aspect, there is provided a method of treating Sjogren's Syndrome in a subject, the method comprising:
administering to a subject in need thereof, a population of T reg cells expressing a binding protein on their surface,
wherein the binding protein comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto; and
wherein the Vβ domain comprises a CDR3 having an amino acid sequence as set forth in SEQ ID NO: 16, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto;
thereby treating the Sjogren's Syndrome in the subject. Preferably, the method may also comprise treating SLE, lupus nephritis and/or autoimmune vasculitis (preferably MPA) in the subject.

In an embodiment of the third aspect of the invention there is also provided a use of a population of T reg cells as described above in the manufacture of a medicament for treating Sjogren's Syndrome in a subject. Preferably, the medicament is also for treating SLE, lupus nephritis and/or MPA in the subject.

In a further embodiment of the third aspect of the invention there is provided a population of T reg cells as described above for use in the treatment of Sjogren's Syndrome, in a subject. Preferably, the use also comprises treating SLE, lupus nephritis and/or MPA in the subject.

In a fourth aspect, there is provided a method of treating autoimmune vasculitis in a subject, the method comprising:
administering to a subject in need thereof, a population of T reg cells expressing a binding protein on their surface,
wherein the binding protein comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto; and
wherein the Vβ domain comprises a CDR3 having an amino acid sequence as set forth in SEQ ID NO: 16, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto;
thereby treating the vasculitis in the subject. Preferably, the vasculitis is microscopic polyangiitis (MPA); granulomatosis with polyangiitis (GPA) or eosinophilic granulomatosis with polyangiitis (EGPA), most preferably MPA. The method may also comprise treating SLE, lupus nephritis and/or Sjogren's Syndrome in the subject.

In an embodiment of the fourth aspect of the invention there is also provided a use of a population of T reg cells as described above in the manufacture of a medicament for treating microscopic polyangiitis (MPA) in a subject. Preferably, the medicament is also for treating SLE, lupus nephritis and/or Sjogren's Syndrome in the subject.

In a further embodiment of the fourth aspect of the invention there is provided a population of T reg cells as described above for use in the treatment of MPA, in a subject. Preferably, the use also comprises treating SLE, lupus nephritis and/or Sjogren's Syndrome in the subject.

In any aspect of the invention, the Sjogren's Syndrome or MPA is characterised by an aberrant immune response to one or more of Smith protein, Ro60 protein and MPO protein in the subject.

In any aspect of the invention, the aberrant immune response comprises the formation of autoantibodies to one or more of the Smith protein, Ro60 protein and MPO protein in the subject.

In any method, use or composition for use of the invention the binding protein preferably comprises:
a Vα domain comprises a CDR3 having an amino acid sequence as set forth in SEQ ID NO: 13, or a sequence having no more than 1, no more than 2, or no more than 3, no more than 4, no more than 5 amino acid substitutions compared to the sequence of SEQ ID NO: 13; and
a Vβ domain comprises a CDR3 having an amino acid sequence as set forth in SEQ ID NO: 16 or a sequence having no more than 1, no more than 2, or no more than 3, no more than 4, no more than 5 amino acid substitutions compared to the sequence of SEQ ID NO: 16.

In any method, use or composition for use of the invention, the binding protein preferably comprises:
a Vα domain comprising a CDR1 having an amino acid sequence as set forth in SEQ ID NO: 11, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto; and
a Vα domain comprising a CDR2 having an amino acid sequence as set forth in SEQ ID NO: 12 or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto; and
a Vβ domain comprising a CDR1 having an amino acid sequence as set forth in SEQ ID NO: 14, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto; and
a Vβ domain comprising a CDR2 having an amino acid sequence as set forth in SEQ ID NO: 15, or a sequence at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical thereto.

In any method, use or composition for use of the invention, the binding protein preferably comprises:
a Vα domain comprising a CDR1 having an amino acid sequence as set forth in SEQ ID NO: 11, or a sequence having no more than 1, no more than 2, or no more than 3 amino acid substitutions compared to the sequence of SEQ ID NO: 11;
a Vα domain comprising a CDR2 having an amino acid sequence as set forth in SEQ ID NO: 12 or a sequence having no more than 1, no more than 2, or no more than 3 amino acid substitutions compared to the sequence of SEQ ID NO: 12;
a Vβ domain comprising a CDR1 having an amino acid sequence as set forth in SEQ ID NO: 14, or a sequence having no more than 1, no more than 2, or no more than 3 amino acid substitutions compared to the sequence of SEQ ID NO: 14;
a Vβ domain comprising a CDR2 having an amino acid sequence as set forth in SEQ ID NO: 15, or a sequence having no more than 1, no more than 2, or no more than 3 amino acid substitutions compared to the sequence of SEQ ID NO: 15; and/or

In any method, use or composition for use of the invention, the binding protein may comprise a Va domain having a CDR1, a CDR2 and a CDR3 comprising the amino acid sequences set forth in SEQ ID NOs: 11, 12 and 13 and a VB domain having a CDR1, a CDR2 and a CDR3 comprising the amino acid sequences set forth in SEQ ID NOs: 14, 15 and 16.

In any method, use or composition for use of the invention as described above, the CDRs are determined according to the IMGT method.

In any method, use or composition for use of the invention, the binding protein may comprise a TCRα chain comprising or consisting of an amino acid sequence as set forth in SEQ ID NO: 23 with at least 1, at least 2, at least 3, at least 4, at least 5, at least 6 and no more than about 10 amino acid insertions, deletions, substitutions, additions or a combination thereof, outside the indicated CDR sequences.

In any method, use or composition for use of the invention, the binding protein may comprise a TCRβ chain comprising or consisting of an amino acid sequence as set forth in SEQ ID NO: 24 with at least 1, at least 2, at least 3, at least 4, at least 5, at least 6 and no more than about 10 amino acid insertions, deletions, substitutions, additions or a combination thereof, outside the indicated CDR sequences.

In any method, use or composition for use of the invention, the binding protein may comprise a TCRα chain comprising or consisting of an amino acid sequence as set forth in SEQ ID NO: 23, and a TCRβ chain comprising or consisting of an amino acid sequence as set forth in SEQ ID NO: 24.

In any method, use or composition for use of the invention, the Smith protein may be SmB/B′, SmD or any other Smith protein described herein. Accordingly, in any aspect the binding protein is capable of binding to SmB/B′ and/or SmD. In one embodiment, the binding protein is capable of binding to a fragment of a Smith protein that comprises or consists of an amino acid sequence of, or equivalent to residues 78-92 of an SmD1 protein or residues 7-21 of a SmB/B′ protein (as set forth in SEQ ID NOs: 1 and 3, respectively).

In any method, use or composition for use of the invention, the binding protein is capable of binding to a complex of a fragment of a Smith protein, Ro60 protein or MPO protein, and an HLA-DR3 and/or an HLA-DR4.5 molecule. Preferably, the binding protein is capable of binding to a fragment of a Smith protein that comprises or consists of an amino acid sequence of, or equivalent to residues 78-92 of an SmD1 protein or residues 7-21 of a SmB/B′ protein (as set forth in SEQ ID NOs: 1 and 3, respectively).

In any method, use or composition for use of the invention, the binding protein is capable of binding to a complex of a fragment of Ro60 protein and an HLA-DR3 molecule, wherein the fragment of the Ro60 protein comprises or consists of an amino acid sequence of, or equivalent to residues 225-239 or residues 369-383 of Ro60, as set forth in SEQ ID NOs: 5 and 6, respectively.

In any method, use or composition for use of the invention, the binding protein is capable of binding to a complex of a fragment of MPO protein and an HLA-DR3 and/or HLA-DR4.5 molecule, wherein the fragment of the MPO protein comprises or consists of an amino acid sequence of, or equivalent to residues 453-467 or residues 724-738 of MPO, as set forth in SEQ ID NOs: 8 and 9, respectively.

In any method, use or composition for use of the invention, the TCRα chain and TCRβ chain of the binding protein, are modified to include a cysteine residue that allows formation of an additional interchain disulfide bond. Preferably, the residue at, or equivalent to, Thr48 on the TCRα chain and the residue at, or equivalent to, Ser57 on the TCRβ chain are replaced with cysteines to facilitate the creation of the additional disulfide bond between the TCR constant regions.

In any method, use or composition for use of the invention, the population of T reg cells may be derived from the subject requiring treatment or from a histocompatible donor. Alternatively, the population of T reg cells is may be derived from stem cells, optionally wherein the stem cells are induced pluripotent stem cells (iPSCs) or embryonic stem cells. In any aspect, the population of T reg cells may be derived from a mixed population of T cells in which a nucleic acid encoding a binding protein as defined herein was introduced.

In any method, use or composition for use of the invention, the population of T regs may be obtained by:
providing a population of T cells exhibiting at least one property of a conventional T cell, optionally wherein the population of T cells is a mixed population of T cells;
introducing a nucleic acid or vector of the invention into the population of T cells, wherein the nucleic acid or vector encodes a binding protein as described herein;
providing conditions to allow the expression of the binding protein on the surface of the T cells,
providing conditions to allow conversion of the population of T cells into T regulatory cells,
   thereby preparing an ex vivo population of T cells exhibiting at least one property of a regulatory T cell, wherein the T cells are specific for one or more of Sm/B′, SmD, Ro60 or MPO proteins.

In another aspect, the present invention provides a method of preparing a population of T regulatory cells for use in the treatment of an autoimmune disease as described herein, the method comprising:
providing a population of T regulatory cells,
introducing a nucleic acid or vector encoding a TCR as described herein, into the population of T regulatory cells,
providing conditions to allow the expression of the TCR on the surface of the T regulatory cells,
thereby preparing a population of T regulatory cells for use in the treatment of an autoimmune disease as described herein.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Dominant reactivity of the promiscuous self-TCR. T cells from a healthy human were co-cultured with monocyte derived dendritic cells (DCs) pulsed with 6 different self-peptides. Each graph shows the number of reactive clones per peptide. Note that only the promiscuous self-TCR sequence is found to be clonally expanded across all peptide stimulations. Regarding the other TCR sequences that are clonally expanded, none of the TCR sequences cross-react with any other peptide, e.g. other TCR sequences that react to SmD1: 78-92 do not react to SmB/B′: 7-21, Ro60: 225-239, Ro60: 369-383, MPO: 453-467 or MPO: 724-738. Solid bars=promiscuous self-TCR sequence. Open bars=other TCR sequences.

FIG. 2: Experimental protocol to demonstrate the efficacy of Ro60-Tregs at halting disease progression in a new humanised model of Sjogren's Syndrome.

FIG. 3: Upregulated Treg genes associated with antigen-specific TCR activation. Upregulated genes associated with TCR activation were FOXP3, CTLA4,IL1R2, CCR10, TIGIT, IKZF2, ITGAM, LGALS1, LGALS3, PI16, IL2RA. Downregulated genes associated Treg were with activation IL13, IFNG, IL7R. These data demonstrate that each epitope can induce promiscuous self-TCR engagement and Treg activation. Solid dots: upregulated genes; grey/shaded dots: down-regulated genes.

FIG. 4: Expression of the promiscuous self-TCR on human Tregs The transduction protocol enables successful expression of the promiscuous self-TCR on the surface of human Tregs. A=mock tranduced human Tregs; B=promiscuous self-TCR transduced T regs. Staining is for GFP expression and VB14 TCR expression.

FIG. 5: Promiscuous self-TCR Tregs bind to peptide-HLA dextramers. Tregs transduced with the promiscuous self-TCR exhibit binding to the SmD1:78-21/HLA-DRB1*03:01, Ro60:369-383/HLA-DRB1*03:01 and MPO:453-567/HLA-DRB1*04:05 dextramers. Solid squares=promiscuous self-TCR transduced Tregs. Open squares=mock transduced T regs.

FIG. 6: Promiscuous self-TCR Tregs are activated following peptide stimulation. Stimulation with SmD1:78-92, Ro60:369-383 and MPO:453-467 induced activation of promiscuous self-TCR transduced Tregs compared to no peptide stimulation. Solid squares=promiscuous self-TCR transduced Tregs. Open squares=mock transduced T regs.

FIG. 7: Promiscuous self-TCR Tregs suppress peptide-specific Tconv cell proliferation. Promiscuous self-TCR transduced Tregs demonstrated more potent suppression of Tconv cell proliferation across all three autoepitopes. Solid squares=promiscuous self-TCR transduced Tregs. Open squares=mock transduced T regs.

FIG. 8: Promiscuous self-TCR engages Sm peptides. CD3 and phalloidin mean pixel intensity at the immune synapse was increased when B-LCLs were pulsed with the Sm epitopes. These data indicate that the promiscuous self-TCR can engage both HLA-DRB1*03:01 restricted Sm epitopes. Arrows indicate immune synapse; BF=bright field; MPI=mean pixel intensity.

### Sequence Information

TABLE 1
Sequences of epitopes and TCRs

| Peptide/ | | | |
|---|---|---|---|
| protein | Description | SEQ ID NO: | Amino acid sequence |
| SmD1 | Epitope | 1 | TLLVDVEPKVKSKKR |
| 78-92 | | | |
| SmD1 | Full protein | 2 | MKLVRFLMKLSHETVTIELKNGTQVHGTITGVDVSMNTHL |
| | | | KAVKMTLKNREPVQLETLSIRGNNIRYFILPDSLPLDTLL |
| | | | VDVEPKVKSKKREAVAGRGRGRGRGRGRGRGRGRGGPRR |
| SmB/B′ | Epitope | 3 | DYRMRCILQDGRIFI |
| 7-21 | | | |
| SmB/B′ | Full protein | 4 | KMLQHIDYRMRCILQDGRIFIGTFKAFDKHMNLILCDCDE |
| | | | FRKIKPKNSKQAEREEKRVLGLVLLRGENLVSMTVEGPP |
| | | | PKDTGIARVPLAGAAGGPGIGRAAGRGIPAGVPMPQAPA |
| | | | GLAGPVRGVGGPSQQVMTPQGRGTVAAAAAAATASIAG |
| | | | APTQYPPGRGGPPPPMGRGAPPPGMMGPPPGMRPPM |
| | | | GPPMGIPPGRGTPMGMPPPGMRPPPPGMRGPPPPGM |
| | | | RPPRP (underlined sequence corresponds |
| | | | to epitope). |
| | | | Note: |
| | | | The sequence above is SmB′, |
| | | | it is an alternatively |
| | | | spliced variant of SmB that |
| | | | only differs at the end |
| | | | (from residue 223: it is “LL” |
| | | | for SmB and “PPPPGMRPPRP” for SmB′. |
| Ro60 225- | Epitope | 5 | LLKYLEAVEKVKRTR |
| 239 | | | |
| Ro60 369- | Epitope | 6 | KRFLLAVDVSASMNQ |
| 383 | | | |
| Ro60 | Full protein | 7 | MEESVNQMQPLNEKQIANSQDGYVWQVTDMNRLHRFLCF |
| | | | GSEGGTYYIKEQKLGLENAEALIRLIEDGRGCEVIQEIK |
| | | | SFSQEGRTTKQEPMLFALAICSQCSDISTKQAAFKAVSE |
| | | | VCRIPTHLFTFIQFKKDLKESMKCGMWGRALRKAIADWY |
| | | | NEKGGMALALAVTKYKQRNGWSHKDLLRLSHLKPSSEGL |
| | | | AIVTKYITKGWKEVHELYKEKALSVETEKLLKYLEAVEK |
| | | | VKRTRDELEVIHLIEEHRLVREHLLTNHLKSKEVWKALL |
| | | | QEMPLTALLRNLGKMTANSVLEPGNSEVSLVCEKLCNEK |
| | | | LLKKARIHPFHILIALETYKTGHGLRGKLKWRPDEEILK |
| | | | ALDAAFYKTFKTVEPTGKRFLLAVDVSASMNQRVLGSIL |
| | | | NASTVAAAMCMVVTRTEKDSYVVAFSDEMVPCPVTTDMT |
| | | | LQQVLMAMSQIPAGGTDCSLPMIWAQKTNTPADVFIVFT |
| | | | DNETFAGGVHPAIALREYRKKMDIPAKLIVCGMTSNGFT |
| | | | IADPDDRGMLDMCGFDTGALDVIRNFTLDMI |
| MPO 453- | Epitope | 8 | MVQIITYRDYLPLVL |
| 467 | | | |
| MPO 724- | Epitope | 9 | PRDFVNCSTLPALNL |
| 738 | | | |
| MPO | Full protein | 10 | MGVPFFSSLRCMVDLGPCWAGGLTAEMKLLLALAGLLAI |
| | | | LATPQPSEGAAPAVLGEVDTSLVLSSMEEAKQLVDKAYK |
| | | | ERRESIKQRLRSGSASPMELLSYFKQPVAATRTAVRAAD |
| | | | YLHVALDLLERKLRSLWRRPFNVTDVLTPAQLNVLSKSS |
| | | | GCAYQDVGVTCPEQDKYRTITGMCNNRRSPTLGASNRAF |
| | | | VRWLPAEYEDGFSLPYGWTPGVKRNGFPVALARAVSNEI |
| | | | VRFPTDQLTPDQERSLMFMQWGQLLDHDLDFTPEPAARA |
| | | | SFVTGVNCETSCVQQPPCFPLKIPPNDPRIKNQADCIPF |
| | | | FRSCPACPGSNITIRNQINALTSFVDASMVYGSEEPLAR |
| | | | NLRNMSNQLGLLAVNQRFQDNGRALLPFDNLHDDPCLLT |
| | | | NRSARIPCFLAGDTRSSEMPELTSMHTLLLREHNRLATE |
| | | | LKSLNPRWDGERLYQEARKIVGAMVQIITYRDYLPLVLG |
| | | | PTAMRKYLPTYRSYNDSVDPRIANVFTNAFRYGHTLIQP |
| | | | FMFRLDNRYQPMEPNPRVPLSRVFFASWRVVLEGGIDPI |
| | | | LRGLMATPAKLNRQNQIAVDEIRERLFEQVMRIGLDLPA |
| | | | LNMQRSRDHGLPGYNAWRRFCGLPQPETVGQLGTVLRNL |
| | | | KLARKLMEQYGTPNNIDIWMGGVSEPLKRKGRVGPLLAC |
| | | | IIGTQFRKLRDGDRFWWENEGVFSMQQRQALAQISLPRI |
| | | | ICDNTGITTVSKNNIFMSNSYPRDFVNCSTLPALNLASW |
| | | | REAS |
| TCR | Description | SEQ ID NO: | Amino acid or nucleotide sequence |
| Promiscuous | CDR1 alpha | 11 | NSASQS |
| TCR (CDRs | (protein) | | |
| defined by | | | |
| IMGT) | | | |
| | CDR2 alpha | 12 | VYSSGN |
| | (protein) | | |
| | CDR3 alpha | 13 | CVVPPGGGFKTIF |
| | (protein) | | |
| | CDR1 beta | 14 | MNHEY |
| | (protein) | | |
| | CDR2 beta | 15 | SMNVEV |
| | (protein) | | |
| | CDR3 beta | 16 | CASRTRTASYGYTF |
| | (protein) | | |
| | CDR1 alpha | 17 | AACAGTGCTTCTCAGTCT |
| | (DNA) | | |
| | CDR2 alpha | 18 | GTATACTCCAGTGGTAAT |
| | (DNA) | | |
| | CDR3 alpha | 19 | TGTGTGGTGCCTCCTGGGGGAGGCTTCAAAACTATCT |
| | (DNA) | | TT |
| | CDR1 beta | 20 | ATGAACCATGAGTAT |
| | (DNA) | | |
| | CDR2 beta | 21 | TCAATGAATGTTGAGGTG |
| | (DNA) | | |
| | CDR3 beta | 22 | TGTGCCAGCAGGACCAGGACAGCCAGTTATGGCTACA |
| | (DNA) | | CCTTC |
| | TCR Variable | 23 | MMISLRVLLVILWLQLSWVWSQRKEVEQDPGPFNVPEG |
| | alpha chain | | ATVAFNCTYSNSASQSFFWYRQDCRKEPKLLMSVYSSG |
| | | | NEDGRFTAQLNRASQYISLLIRDSKLSDSATYLCVVPP |
| | | | GGGFKTIFGAGTRLFVKANIQNPDPAVYQLRDSKSSDK |
| | | | SVCLFTDFDSQTNVSQSKDSDVYITDKTVLDMRSMDFK |
| | | | SNSAVAWSNKSDFACANAF |
| | TCR Variable | 24 | MGPQLLGYVVLCLLGAGPLEAQVTQNPRYLITVTGKKL |
| | bets chain | | TVTCSQNMNHEYMSWYRQDPGLGLRQIYYSMNVEVTDK |
| | | | GDVPEGYKVSRKEKRNFPLILESPSPNQTSLYFCASRT |
| | | | RTASYGYTFGSGTRLTVVEDLNKVFPPEVAVFEPSEAE |
| | | | ISHTQKATLVCLATGFFPDHVELSWWVNGKEVHSGVST |
| | | | DPQPLKEQPALNDSRYCL |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The inventors have identified a promiscuous self-epitope T cell receptor (self-TCR) which is able to bind to a number of autoantigens associated with autoimmune disease, in particular: Smith protein, Ro60 protein and myeloperoxidase (MPO) protein.

Autoantibodies to Smith, Ro60 and MPO proteins are associated with various autoimmune diseases, including systemic lupus erythematosus (SLE), Sjogren's Syndrome and autoimmune vasculitis, especially microscopic polyangiitis and eosinophilic granulomatosis with polyangiitis (Churg Strauss syndrome). Thus, the present invention relates to a single TCR for use in the treatment of a number of autoimmune diseases.

A particular advantage of the present invention is that it provides a single product (e.g, population of Treg cells expressing the promiscuous TCR), for use in treating a broader group of individuals suffering from autoimmune disease, while being specific for the particular auto-antigens associated with their disease. This is in contrast to existing treatments applied across patient groups, wherein the treatments are non-specific and therefore likely to be less efficacious and potentially result in increased side-effect. The approach of the present invention allows for the specific targeting of the source of autoimmune disease in more than one disease population.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4,IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, T cell receptors and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989; Al-Lazikani et al., J Mol Biol 273, 927-948, 1997; or the IMGT system discussed in Giudicelli et al., Nucleic Acids Res. 25:206-211 1997.

In preferred embodiments, the CDRs as defined herein are defined according to the IMGT method.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 1 to 15” will be understood in an inclusive manner, i.e., the region comprises a sequence of amino acids as numbered 1, 2, 3, 4, 5, 6, 7, 8, 9, 10, 11, 12, 13, 14 and 15 in a specified sequence.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino-or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “nucleic acid” or “nucleic acid molecule” refers to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., a-enantiomeric forms of naturally-occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded.

As used herein, the term “recombinant” refers to a cell, microorganism, nucleic acid molecule, or vector that has been genetically engineered by human intervention-that is, modified by introduction of an exogenous or heterologous nucleic acid molecule, or refers to a cell or microorganism that has been altered such that expression of an endogenous nucleic acid molecule or gene is controlled, deregulated or constitutive. Human generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

### Binding Proteins

A “binding protein” as used herein, refers to a proteinaceous molecule or portion thereof (e.g., peptide, oligopeptide, polypeptide, protein) that possesses the ability to specifically and non-covalently associate, unite, or combine with a target (e.g., Smith protein or fragment thereof, Smith protein fragment:MHC complex). A binding protein may be purified, substantially purified, synthetic or recombinant. Exemplary binding proteins include single chain immunoglobulin variable regions (e.g., scTCR, scFv).

In certain embodiments, any of the binding proteins of the invention are each a T cell receptor (TCR), a chimeric antigen receptor or an antigen-binding fragment of a TCR, any of which can be chimeric, humanized or human. In further embodiments, an antigen-binding fragment of the TCR comprises a single chain TCR (scTCR) or a chimeric antigen receptor (CAR). In certain embodiments, a binding protein is a TCR.

“T cell receptor” (TCR) refers to an immunoglobulin superfamily member (having a variable binding domain, a constant domain, a transmembrane region, and a short cytoplasmic tail; see, e.g., Janeway et al., Immunobiology: The Immune System in Health and Disease, 3rd Ed., Current Biology Publications, p. 4:33, 1997) capable of specifically binding to an antigen peptide bound to a MHC receptor. A TCR can be found on the surface of a cell or in soluble form and generally is comprised of a heterodimer having α (alpha) and β (beta) chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin domains, a variable domain (e.g., α-chain variable domain or Vα, β-chain variable domain or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) at the N-terminus, and one constant domain (e.g., α-chain constant domain or Cα, typically amino acids 117 to 259 based on Kabat, β-chain constant domain or Cβ, typically amino acids 117 to 295 based on Kabat) adjacent to the cell membrane. Also like immunoglobulins, the variable domains contain complementary determining regions (CDRs) separated by framework regions (FRs) (see, e.g., Jores et al., Proc. Nat'l Acad. Sci. U.S.A. 57:9138, 1990; Chothia et al, EMBO J. 7:3745, 1988; see also Lefranc et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, rabbit or other mammal.

In any of the aforementioned embodiments, the present disclosure provides for the use of a high affinity engineered T cell receptor (TCR), comprising an alpha-chain (α-chain) and a beta-chain (β-chain), wherein the TCR binds to a complex of a fragment of a Smith protein, Ro60 protein or MPO protein and an HLA-DR3 or HLA-DR4 molecule. In certain embodiments, a V beta chain comprises or is derived from a TRBV3, TRBV4, TRBV5, TRBV6, TRBV7, TRBV11, TRBV19, TRBV20, TRBV24, or TRBV28 allele. In further embodiments, a V alpha chain comprises or is derived from a TRAV1, TRAV2, TRAV3, TRAV4, TRAV8, TRAV9, TRAV12, TRAV14, TRAV17, TRAV21, TRAV23, TRAV25, TRAV26, TRAV27, TRAV29, TRAV38, TRAV39, or TRAV40 allele. In particular embodiments, a binding protein for use according to the invention comprises: (a) a V beta chain that comprises or is derived from a TRBV11allele (preferably TRBV11-2) and a V alpha chain that comprises or is derived from a TRAV9 allele (preferably TRAV9-2); (b) a V beta chain that comprises or is derived from a TRBV6 allele (preferably TRBV6-1) and a V alpha chain that comprises or is derived from a TRAV25 allele; (c) a V beta chain that comprises or is derived from a TRBV7allele (preferably TRBV7-9) and a V alpha chain that comprises or is derived from a TRAV29 allele; (d) a V beta chain that comprises or is derived from a TRBV28 allele and a V alpha chain that comprises or is derived from a TRAV23 allele; (e) a V beta chain that comprises or is derived from a TRBV7 allele (preferably TRVB7-9) and a V alpha chain that comprises or is derived from a TRAV26 allele (preferably TRAV26-1); (f) a V beta chain that comprises or is derived from a TRBV7 allele (preferably TRBV7-3) and a V alpha chain that comprises or is derived from a TRAV8 allele (preferably TRAV8-6); (g) a V beta chain that comprises or is derived from a TRBV20 allele (preferably TRBV20-1) and a V alpha chain that comprises or is derived from a TRAV9allele (preferably TRAV9-2); (h) a V beta chain that comprises or is derived from a TRBV3 allele (preferably TRBV3-1) and a V alpha chain that comprises or is derived from a TRAV2 allele; (i) a V beta chain that comprises or is derived from a TRBV4 allele (preferably TRBV4-2) and a V alpha chain that comprises or is derived from a TRAV17allele; (j) a V beta chain that comprises or is derived from a TRBV4 allele (preferably TRBV4-2) and a V alpha chain that comprises or is derived from a TRAV27 allele; (k) a V beta chain that comprises or is derived from a TRBV6 allele (preferably TRBV6-5) and a V alpha chain that comprises or is derived from a TRAV2 allele.

In further embodiments, a binding protein for use according to the invention comprises: (a) a V beta chain that comprises or is derived from a TRBV20 allele (preferably TRBV20-1) and a V alpha chain that comprises or is derived from a TRAV38allele (preferably TRAV38-1); (b) a V beta chain that comprises or is derived from a TRBV6 allele (preferably TRBV6-4) and a V alpha chain that comprises or is derived from a TRAV1 allele (preferably TRAV1-2); (c) a V beta chain that comprises or is derived from a TRBV6 allele (preferably TRBV6-4) and a V alpha chain that comprises or is derived from a TRAV4 allele; (d) a V beta chain that comprises or is derived from a TRBV4 allele (preferably TRBV4-1) and a V alpha chain that comprises or is derived from a TRAV17 allele; (e) a V beta chain that comprises or is derived from a TRBV5allele (preferably TRBV5-4) and a V alpha chain that comprises or is derived from a TRAV21 allele; (f) a V beta chain that comprises or is derived from a TRBV28 allele and a V alpha chain that comprises or is derived from a TRAV27 allele; (g) a V beta chain that comprises or is derived from a TRBV24 allele (preferably TRBV24-1) and a V alpha chain that comprises or is derived from a TRAV1 allele (preferably TRAV1-1).

In any aspect or embodiment, the binding protein for use according to the invention comprises (a) a Vbeta chain that comprises or is derived from a TRBJ2 allele (preferably TRBJ2-7) and a Valpha chain that comprises or is derived from a TRAJ47allele; (b) a Vbeta chain that comprises or is derived from a TRBJ2 allele (preferably TRBJ2-3) and a Valpha chain that comprises or is derived from a TRAJ54 allele; (c) a Vbeta chain that comprises or is derived from a TRBJ1 allele (preferably TRBJ1-1) and a Valpha chain that comprises or is derived from a TRAJ48 allele; (d) a Vbeta chain that comprises or is derived from a TRBJ2 allele (preferably TRBJ2-1) and a Valpha chain that comprises or is derived from a TRAJ44 allele; (e) a Vbeta chain that comprises or is derived from a TRBJ1 allele (preferably TRBJ1-5) and a Valpha chain that comprises or is derived from a TRAJ38 allele; (f) Vbeta chain that comprises or is derived from a TRBJ2 allele (preferably TRBJ2-1) and a Valpha chain that comprises or is derived from a TRAJ22 allele; (g) a Vbeta chain that comprises or is derived from a TRBJ2 allele (preferably TRBJ2-7) and a Valpha chain that comprises or is derived from a TRAJ11allele; (h) a Vbeta chain that comprises or is derived from a TRBJ1 allele (preferably TRBJ1-1) and a Valpha chain that comprises or is derived from a TRAJ8 allele; (i) a Vbeta chain that comprises or is derived from a TRBJ2 allele (preferably TRBJ2-3) and a Valpha chain that comprises or is derived from a TRAJ45 allele; (j) a Vbeta chain that comprises or is derived from a TRBJ2 allele (preferably TRBJ2-3) and a Valpha chain that comprises or is derived from a TRAJ49 allele; (k) a Vbeta chain that comprises or is derived from a TRBJ1 allele (preferably TRBJ1-2) and a Valpha chain that comprises or is derived from a TRAJ7 allele.

In any aspect or embodiment, the binding protein for use according to the invention comprises (a) a Vbeta chain that comprises or is derived from a TRBJ1 allele (preferably TRBJ1-4) and a Valpha chain that comprises or is derived from a TRAJ48allele; (b) a Vbeta chain that comprises or is derived from a TRBJ1 (preferably TRBJ1-5) allele and a Valpha chain that comprises or is derived from a TRAJ48 allele; (c) a Vbeta chain that comprises or is derived from a TRBJ2 allele (preferably TRBJ2-1) and a Valpha chain that comprises or is derived from a TRAJ48 allele; (d) a Vbeta chain that comprises or is derived from a TRBJ2 allele (preferably TRBJ2-7) and a Valpha chain that comprises or is derived from a TRAJ48 allele; (e) a Vbeta chain that comprises or is derived from a TRBJ1 allele (preferably TRBJ1-1) and a Valpha chain that comprises or is derived from a TRAJ88 allele; (f) a Vbeta chain that comprises or is derived from a TRBJ1 allele (preferably TRBJ1-2) and a Valpha chain that comprises or is derived from a TRAJ12 allele; (g) a Vbeta chain that comprises or is derived from a TRBJ1 allele (preferably TRBJ1-2) and a Valpha chain that comprises or is derived from a TRAJ3allele; (h) Vbeta chain that comprises or is derived from a TRBJ1 allele (preferably TRBJ1-3) and a Valpha chain that comprises or is derived from a TRAJ9 allele; (i) a Vbeta chain that comprises or is derived from a TRBJ2 allele (preferably TRBJ2-1) and a Valpha chain that comprises or is derived from a TRAJ28 allele; (j) a Vbeta chain that comprises or is derived from a TRBJ1 allele (preferably TRBJ1-2) and a Valpha chain that comprises or is derived from a TRAJ41 allele; (k) a Vbeta chain that comprises or is derived from a TRBJ2 allele (preferably TRBJ2-1) and a Valpha chain that comprises or is derived from a TRAJ9 allele.

In any of the aforementioned embodiments, the present disclosure provides a high affinity engineered T cell receptor (TCR), comprising an alpha-chain (α-chain) and a beta-chain (β-chain), wherein the TCR binds to a complex of a fragment of a Smith protein or Ro60 protein and an HLA-DR3 molecule, preferably, the HLA-DR3 molecule is an HLA-DRA*01:01 and HLA-DRB1*03:01 molecule. In certain embodiments, a V beta chain comprises or is derived from a TRB2, TRBV4, TRBV5, TRB6, TRB7, TRBV9,TRB10, TRBV11, TRB12, TRBV20, TRBV24, TRB27 or TRBV29 allele. In further embodiments, a V alpha chain comprises or is derived from a TRAV1, TRAV2, TRAV8,TRAV9, TRAV10, TRAV12, TRAV20, TRAV26, TRAV30 or TRAV36 allele.

In particular embodiments, a binding protein for use according to the invention comprises: (a) a V beta chain that comprises or is derived from a TRBV5 allele (preferably TRBV5-1) and a V alpha chain that comprises or is derived from a TRAV20allele; (b) a V beta chain that comprises or is derived from a TRBV29 allele (preferably TRBV29-1) and a V alpha chain that comprises or is derived from a TRAV12 allele (preferably TRAV12-1); (c) a V beta chain that comprises or is derived from a TRBV4allele (preferably TRBV4-1) and a V alpha chain that comprises or is derived from a TRAV26 allele (preferably TRAV26-2); (d) a V beta chain that comprises or is derived from a TRBV4 allele (preferably TRBV4-1) and a V alpha chain that comprises or is derived from a TRAV30 allele; (e) a V beta chain that comprises or is derived from a TRBV4 allele (preferably TRVB4-1) and a V alpha chain that comprises or is derived from a TRAV36 allele (preferably TRAV36DV7); (f) a V beta chain that comprises or is derived from a TRBV24 allele (preferably TRBV24-1) and a V alpha chain that comprises or is derived from a TRAV12 allele (preferably TRAV12-1); (g) a V beta chain that comprises or is derived from a TRBV11 allele (preferably TRBV11-2) and a V alpha chain that comprises or is derived from a TRAV12 allele (preferably TRAV12-3); (h) a V beta chain that comprises or is derived from a TRBV20 allele (preferably TRBV20-1) and a V alpha chain that comprises or is derived from a TRAV9 allele (preferably TRAV9-2); (i) a V beta chain that comprises or is derived from a TRBV9 allele and a V alpha chain that comprises or is derived from a TRAV9 allele (preferably TRAV9-2); (j) a V beta chain that comprises or is derived from a TRBV20 allele (preferably TRBV20-1) and a V alpha chain that comprises or is derived from a TRAV12 allele (preferably TRAV12-1).

In further embodiments, a binding protein for use according to the invention comprises: (a) a V beta chain that comprises or is derived from a TRBV27 allele and a V alpha chain that comprises or is derived from a TRAV12 allele (preferably TRAV12-1); (b) a V beta chain that comprises or is derived from a TRBV6 allele (preferably TRBV6-1) and a V alpha chain that comprises or is derived from a TRAV1 allele (preferably TRAV1-2); (c) a V beta chain that comprises or is derived from a TRBV7 allele (preferably TRBV7-9) and a V alpha chain that comprises or is derived from a TRAV12allele (preferably TRAV12-2); (d) a V beta chain that comprises or is derived from a TRBV2 allele and a V alpha chain that comprises or is derived from a TRAV8 allele (TRAV8-3); (e) a V beta chain that comprises or is derived from a TRBV8 allele (preferably TRBV8-3) and a V alpha chain that comprises or is derived from a TRAV5 allele (preferably TRAV5-1); (f) a V beta chain that comprises or is derived from a TRBV7 allele (preferably TRBV7-9) and a V alpha chain that comprises or is derived from a TRAV10 allele; (g) a V beta chain that comprises or is derived from a TRBV7allele (preferably TRBV7-9) and a V alpha chain that comprises or is derived from a TRAV19 allele; (h) a V beta chain that comprises or is derived from a TRBV10 allele (preferably TRBV10-3) and a V alpha chain that comprises or is derived from a TRAV2allele; (i) a V beta chain that comprises or is derived from a TRBV12 allele (preferably TRBV12-4) and a V alpha chain that comprises or is derived from a TRAV20 allele.

In certain embodiments, a V beta chain comprises or is derived from a TRBJ1or TRBJ2 allele. In further embodiments, a V alpha chain comprises or is derived from a TRAJ3, TRAJ6, TRAJ9, TRAJ13, TRAJ17, TRAJ23, TRAJ27, TRAJ28, TRAJ31,TRAJ33, TRAJ37, TRAJ42, TRAJ45, TRAJ47, TRAJ48, TRAV49 or TRAV54 allele.

In particular embodiments, the binding protein for use according to the invention comprises (a) a Vbeta chain that comprises or is derived from a TRBJ1 allele (preferably TRBJ1-1) and a Valpha chain that comprises or is derived from a TRAJ6allele; (b) a Vbeta chain that comprises or is derived from a TRBJ1 allele (preferably TRBJ1-5) and a Valpha chain that comprises or is derived from a TRAJ45 allele; (c) a Vbeta chain that comprises or is derived from a TRBJ2 allele (preferably TRBJ2-2) and a Valpha chain that comprises or is derived from a TRAJ54 allele; (d) a Vbeta chain that comprises or is derived from a TRBJ2 allele (preferably TRBJ2-1) and a Valpha chain that comprises or is derived from a TRAJ28 allele; (e) a Vbeta chain that comprises or is derived from a TRBJ2 allele (preferably TRBJ2-1) and a Valpha chain that comprises or is derived from a TRAJ49 allele; (f) Vbeta chain that comprises or is derived from a TRBJ2 allele (preferably TRBJ2-2) and a Valpha chain that comprises or is derived from a TRAJ48 allele; (g) a Vbeta chain that comprises or is derived from a TRBJ1 allele (preferably TRBJ1-2) and a Valpha chain that comprises or is derived from a TRAJ17allele; (h) a Vbeta chain that comprises or is derived from a TRBJ2 allele (preferably TRBJ2-7) and a Valpha chain that comprises or is derived from a TRAJ27 allele; (i) a Vbeta chain that comprises or is derived from a TRBJ2 allele (preferably TRBJ2-1) and a Valpha chain that comprises or is derived from a TRAJ37 allele; (j) a Vbeta chain that comprises or is derived from a TRBJ2 allele (preferably TRBJ2-1) and a Valpha chain that comprises or is derived from a TRAJ3 allele.

In particular embodiments, the binding protein for use according to the invention comprises (a) a Vbeta chain that comprises or is derived from a TRBJ1 allele (preferably TRBJ1-2) and a Valpha chain that comprises or is derived from a TRAJ9allele; (b) a Vbeta chain that comprises or is derived from a TRBJ2 (preferably TRBJ2-7) allele and a Valpha chain that comprises or is derived from a TRAJ33 allele; (c) a Vbeta chain that comprises or is derived from a TRBJ2 allele (preferably TRBJ2-1) and a Valpha chain that comprises or is derived from a TRAJ49 allele; (d) a Vbeta chain that comprises or is derived from a TRBJ2 allele (preferably TRBJ2-6) and a Valpha chain that comprises or is derived from a TRAJ13 allele; (e) a Vbeta chain that comprises or is derived from a TRBJ1 allele (preferably TRBJ1-1) and a Valpha chain that comprises or is derived from a TRAJ23 allele; (f) a Vbeta chain that comprises or is derived from a TRBJ2 allele (preferably TRBJ2-1) and a Valpha chain that comprises or is derived from a TRAJ47allele; (g) a Vbeta chain that comprises or is derived from a TRBJ2 allele (preferably TRBJ2-7) and a Valpha chain that comprises or is derived from a TRAJ42allele; (h) Vbeta chain that comprises or is derived from a TRBJ2 allele (preferably TRBJ2-1) and a Valpha chain that comprises or is derived from a TRAJ47 allele; (i) a Vbeta chain that comprises or is derived from a TRBJ2 allele (preferably TRBJ2-5) and a Valpha chain that comprises or is derived from a TRAJ31 allele; (j) a Vbeta chain that comprises or is derived from a TRBJ1 allele (preferably TRBJ1-4) and a Valpha chain that comprises or is derived from a TRAJ47 allele.

In any aspect or embodiment, the binding protein of the invention comprises a V beta chain that comprises or is derived from a TRBD1 or TRBD2 allele.

In any aspect or embodiment, the binding protein of the invention comprises a V beta chain that comprises or is derived from a TRC1 or TRBC2 allele and a V alpha chain that comprises or is derived from a TRAC allele.

In any aspect of the present invention, the binding protein comprises a Vα chain comprising the Vα domain and a Vβ chain comprising a Vβ domain. Preferably, the Vα chain and Vβ chain are modified to include a cysteine residue that allows formation of an additional interchain disulfide bond. The cysteine introduced into each of the Vα chain and Vβ chains allows preferential pairing of the Vα and Vβ chain when expressed in a cell that expresses endogenous TCR Vα and Vβ chains. Preferably, the residue at, or equivalent to, Thr48 on the TCR α chain and the residue at, or equivalent to, Ser57 on the TCR β chain are replaced with cysteines to facilitate the creation of an additional disulfide bond between the TCR constant regions. This modification allows preferential pairing of the introduced TCRs and reduces mispairing with endogenous TCRs. This is particularly beneficial for adoptive cell therapies where T regulatory cells are modified to express exogenous TCRs.

Methods useful for isolating and purifying recombinantly produced soluble TCR, by way of example, may include obtaining supernatants from suitable host cell/vector systems that secrete the recombinant soluble TCR into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble TCR described herein include batch cell culture, which is monitored and controlled to maintain appropriate culture conditions. Purification of the soluble TCR may be performed according to methods described herein and known in the art.

The SmB/B′-specific binding proteins or domains as described herein (e.g., SEQ ID NOS.: 11-24, and variants thereof), may be functionally characterized according to any of a large number of art accepted methodologies for assaying T cell activity, including determination of T cell binding, activation or induction and also including determination of T cell responses that are antigen-specific. Examples include determination of T cell proliferation, T cell cytokine release, antigen specific T cell stimulation, MHC restricted T cell stimulation, CTL activity (e.g., by detecting Cr release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, MA (1986); Mishell and Shigii (eds.) Selected Methods in Cellular Immunology, Freeman Publishing, San Francisco, CA (1979); Green and Reed, Science 281:1309(1998) and references cited therein.

The Sm (Smith) and related nuclear ribonucleoproteins (nRNPs) are targets for autoantibodies in SLE. These antigens are present in subcellular organelles called spliceosomes that are composed of peptide containing small RNAs. Anti-Sm antibodies are present in 15 to 30% of the patients with SLE, but they are highly specific for SLE. They occur more frequently (60%) in young black females with SLE. They almost never occur in healthy individuals or patients with other diseases. Anti-Sm antibodies are not to be confused with anti-smooth muscle antibodies detected in autoimmune liver disease.

The Sm and nuclear ribonucleoprotein (RNP) antigens are a particulate complex composed of small nuclear RNAs (U-RNAs) and proteins. This complex has also been referred to as extractable nuclear antigens (ENA), since it is soluble in saline. Autoantibodies to these antigens occur in systemic lupus erythematosis and mixed connective tissue disease. Among the proteins present in the complex are the “SmB/B” and “SmD” proteins.

As used herein, SmB/B′ refers to the ribonucleoprotein termed “small nuclear ribonucleoprotein-associated protein B and B”, a protein that in humans is encoded by the SNRPB gene. SmB/B′ may also be referred to by the aliases: COD, SNRPB1,snRNP-B, CCMS and small nuclear ribonucleoprotein polypeptides B and B1.

The protein encoded by the SNRPB gene is one of several nuclear proteins that are found in common among U1, U2, U4/U6, and U5 small ribonucleoprotein particles (snRNPs). These snRNPs are involved in pre-mRNA splicing, and the encoded protein may also play a role in pre-mRNA splicing or snRNP structure. Two transcript variants encoding different isoforms (B and B′) have been found for this gene.

All of the nine core proteins of the Sm complex, but most frequently the B and D polypeptides, are targets of the anti-Sm autoimmune response.

As used herein “Ro60” refers to 60 kDa SS-A/Ro ribonucleoprotein, an RNA-binding protein. The Ro 60 kDa autoantigen is a major target of the immune response in patients suffering from two systemic rheumatic diseases, systemic lupus erythematosus and Sjogren's syndrome. In lupus patients, anti-Ro antibodies are associated with photosensitive skin lesions and with neonatal lupus, a syndrome in which mothers with anti-Ro antibodies give birth to children with photosensitive skin lesions and a cardiac conduction defect, third degree heart block. In vertebrate cells, the Ro protein binds small RNAs of unknown function known as Y RNAs. Although the cellular function of Ro has long been mysterious, recent studies have implicated Ro in two distinct processes: small RNA quality control and the enhancement of cell survival following exposure to ultraviolet irradiation. Most interestingly, mice lacking the Ro protein develop an autoimmune syndrome that shares some features with systemic lupus erythematosus in patients, suggesting that the normal function of Ro may be important for the prevention of this autoimmune disease.

As used herein, myeloperoxidase (MPO) is a is a peroxidase enzyme that in humans is encoded by the MPO gene on chromosome 17. MPO is most abundantly expressed in neutrophil granulocytes and produces hypohalous acids including hypochlorous acid. It is a lysosomal protein stored in azurophilic granules of the neutrophil and released into the extracellular space during degranulation. Antibodies against MPO are implicated in various types of vasculitis, most prominently three clinically and pathologically recognized forms: granulomatosis with polyangiitis (GPA), microscopic polyangiitis (MPA); and eosinophilic granulomatosis with polyangiitis (EGPA).

### Cells for Administration Including Adoptive Cell Therapy

The present invention provides methods of treatment, wherein the methods comprise administering cells, particularly Treg cells expressing a TCR as described herein. The Treg cells may be provided in a population of T cells, or in a mixed population of cells.

In certain embodiments, nucleic acid molecules encoding a binding protein as described herein are used to transfect/transduce a host cell (e.g., Treg cells) for use in adoptive transfer therapy. Nucleic acids encoding said binding proteins are described elsewhere in this document.

The present invention provides a method of preparing a population of T regulatory cells for use in the treatment of an autoimmune disease characterised by an aberrant immune response to one or more of SmB/B′, SmD, Ro60, and MPO, the method comprising:
providing a population of T regulatory cells,
introducing a nucleic acid or vector encoding a TCR as described herein, into the population of T regulatory cells,
providing conditions to allow the expression of the TCR on the surface of the T regulatory cells,
thereby preparing a population of T regulatory cells for use in the treatment of an autoimmune disease characterised by an aberrant immune response to one or more of SmB/B′, SmD, Ro60, and MPO.

The present invention also provides a method for treating an autoimmune disease characterised by an aberrant immune response to one or more of SmB/B′, SmD, Ro60, and MPO in a subject, the method comprising:
administering to a subject an effective amount of T regulatory cells that express on their surface a binding protein comprising a T cell receptor (TCR) α-chain variable (Vα or Valpha) domain and a TCR β-chain variable (Vβ or Vbeta) domain, wherein the binding protein is as described herein,
thereby treating the autoimmune disease characterised by an aberrant immune response to one or more of SmB/B′, SmD, Ro60, and MPO in the subject.

Further, the present invention relates to a method for preparing an ex vivo population of T cells specific to SmB/B′, SmD, Ro60, and MPO and exhibiting at least one property of a regulatory T cell, the method comprising:
providing a population of T cells exhibiting at least one property of a regulatory T cell,
introducing a nucleic acid or vector as described herein into the population of T cells, wherein the nucleic acid or vector encodes a binding protein as described herein;
providing conditions to allow the expression of the binding protein on the surface of the T cells,
thereby preparing an ex vivo population of T cells specific to SmB/B′, SmD, Ro60, and MPO and exhibiting at least one property of a regulatory T cell. Preferably T cells exhibiting at least one property of a regulatory T cell are derived from a biological sample from a subject having SLE.

The T cells exhibiting at least one property of a regulatory T cell used in a method or use of the invention may be selected from healthy subjects. The T cells may be isolated from a histocompatible donor.

The T cells may be reprogrammed from somatic cells, or may be differentiated from iPSCs or other stem cells.

In another aspect, the present invention provides a method of treating or preventing a condition in a subject, wherein the condition is associated with an aberrant, unwanted or otherwise inappropriate immune response to SmB/B′, SmD, Ro60 or MPO proteins, the method comprising:
providing a population of T cells exhibiting at least one property of a regulatory T cell,
introducing a nucleic acid or vector encoding a binding protein as described herein, into the population of T cells,
providing conditions to allow the expression of the binding protein on the surface of the T cells,
administering the T cells expressing the binding protein on their surface,
thereby treating or preventing the condition in the subject.

In alternative embodiments, the present invention provides a method of preparing an ex vivo population of T cells specific to SmB/B′, SmD, Ro60 and MPO proteins, and exhibiting at least one property of a regulatory T cell, the method comprising:
providing a population of T cells exhibiting at least one property of a conventional T cell, optionally wherein the population of T cells is a mixed population of T cells;
introducing a nucleic acid or vector into the population of T cells, wherein the nucleic acid or vector encodes a binding protein as described herein;
providing conditions to allow the expression of the binding protein on the surface of the T cells,
providing conditions to allow conversion of the population of T cells into T regulatory cells,
thereby preparing an ex vivo population of T cells exhibiting at least one property of a regulatory T cell and which are specific to SmB/B′, SmD, Ro60 and MPO proteins.

In any embodiment, the conditions for allowing conversion of a conventional T cell or mixed population of T cells, into a T regulatory cell may comprise contacting the conventional T cells or mixed population of T cells with one or more agents, or increasing the expression of one or more factors suitable for conversion of conventional T cells into regulatory T cells. The one or more agents or factors may comprise: TGF-β, Foxp3 or an agent for increasing expression thereof.

In certain embodiments, one or more peptides described herein derived from SmB/B′, SmD, Ro60, and MPO, and listed in Table 1, may be used to activate and//or expand a population of T cells, in order to generate T cells (e.g., Treg cells) having specificity for the peptides. For example, the present invention provides a method of preparing a population of T regulatory cells for use in the treatment of an autoimmune disease as described herein, the method comprising:
i) culturing a population of T regulatory cells in the presence of a first peptide selected from the SmD, SmB/B′, Ro60 or MPO peptides recited in Table 1 herein, under conditions and for a sufficient time to allow expansion of a subpopulation which are activated by the first peptide,
ii) culturing the subpopulation of T regulatory cells obtained in i) in the presence of a second peptide selected from the SmD, SmB/B′, Ro60 or MPO peptides recited in Table 1 herein, under conditions and for a sufficient time to allow expansion of a subpopulation which are activated by the first and second peptides,
thereby preparing a population of T regulatory cells for use in the treatment of an autoimmune disease as described herein. Optionally, the methods further include steps including culturing the subpopulation of T regulatory cells activated by the first and second peptides, in the presence of a third or fourth peptide selected from the SmD, SmB/B′, Ro60 or MPO peptides recited in Table 1 herein, thereby obtaining a subpopulation of Tregs which are activated by all four peptides. The first and second peptides being different, for example derived from a different protein (and when the method includes a third or fourth peptide, those are derived from different proteins to the proteins that the first and second peptides are derived from).

In one embodiment, the first peptide is derived from Ro60 protein (for example, the peptide may comprise or consist of the amino acid sequence as set forth in SEQ ID NO: 5, 6 or 7) and the second peptide is derived from Smith protein (for example, the peptide may comprise or consist of the amino acid sequence as set forth in SEQ ID NO: 1, 2, 3 or 4). Optionally, the third peptide is derived from MPO protein (for example, the peptide may comprise or consist of the amino acid sequence as set forth in SEQ ID NO: 8, 9 or 10), or when the second peptide is derived from SmD1 (for example the peptide may comprise or consist of the amino acid sequence as set forth in SEQ ID NO: 1 or 2), the third peptide may be derived from SmB/B′ (for example, the peptide may comprise or consist of the amino acid sequence as set forth in SEQ ID NO: 3 or 4).

In one embodiment, the first peptide is derived from Ro60 protein (for example, the peptide may comprise or consist of the amino acid sequence as set forth in SEQ ID NO: 5, 6 or 7) and the second peptide is derived from MPO protein (for example, the peptide may comprise or consist of the amino acid sequence as set forth in SEQ ID NO: 8, 9 or 10). Optionally, the third peptide is Smith protein (for example, the peptide may comprise or consist of the amino acid sequence as set forth in SEQ ID NO: 1, 2, 3 or 4).

In one embodiment, the first peptide is derived from Smith protein (for example, the peptide may comprise or consist of the amino acid sequence as set forth in SEQ ID NO: 1, 2, 3 or 4) and the second peptide is derived from MPO protein (for example, the peptide may comprise or consist of the amino acid sequence as set forth in SEQ ID NO: 8, 9 or 10). Optionally, the third peptide is derived from Ro60 protein (for example, the peptide may comprise or consist of the amino acid sequence as set forth in SEQ ID NO: 5, 6 or 7) or when the first peptide is derived from SmD1 (for example the peptide may comprise or consist of the amino acid sequence as set forth in SEQ ID NO: 1 or 2), the third peptide may be derived from SmB/B′ (for example, the peptide may comprise or consist of the amino acid sequence as set forth in SEQ ID NO: 3 or 4).

Advances in TCR sequencing have been described (e.g., Robins et al, Blood 114:4099, 2009; Robins etal, Sci. Translat. Med. 2:47ra64, 2010; Robins et al, (September 10) J. 1 mm. Meth. Epub ahead of print, 2011; Warren et al, Genome Res. 2 1:790, 2011) and may be employed in the course of practicing the embodiments according to the present disclosure. Similarly, methods for transfecting/transducing T cells with desired nucleic acids have been described (e.g., U.S. Patent Application Pub. No. US 2004/0087025) as have adoptive transfer procedures using T-cells of desired antigen-specificity (e.g., Schmitt et al, Hum. Gen. 20:1240, 2009; Dossett etal, Mol. Ther. 77:742, 2009; Till et al, Blood 772:2261, 2008; Wang et al, Hum. Gene Ther. 75:712, 2007; Kuball et al, Blood 709:2331, 2007; US 2011/0243972; US 2011/0189141; e n et al, Ann. Rev. Immunol. 25:243, 2007), such that adaptation of these methodologies to the presently disclosed embodiments is contemplated, based on the teachings herein, including those directed to binding proteins of the invention.

A population of cells comprising regulatory T (Treg) cells may be derived from any source in which Treg cells exist, such as peripheral blood, the thymus, lymph nodes, spleen, and bone marrow.

A population of cells comprising Treg cells may also be derived from a mixed population of T cells, or from a population of conventional T cells. As described herein, the mixed population or conventional T cells may be contacted with a peptide of the invention to enrich Sm antigen specificity in the T cells. Alternatively, the mixed population or conventional T cells may be transduced with a nucleic acid encoding a binding protein of the invention. The T cells may then be converted into Treg cells using standard techniques known to the skilled person for generation of Treg cells. In certain embodiments, the mixed population of T cells, or conventional T cells are cultured in conditions to allow for increased expression of TGF-beta, Foxp3. This includes culturing cells with anti-CD3/anti-CD28 antibodies, inhibition of CDK8/19 high doses of IL-2, TGF-beta, and rapamycin In further embodiments, the converted or enriched population of Treg cells are stabilised (for example, by contacting the cells with Vitamin C or other agent for stabilising the Tregs).

The Treg cells used for infusion (or indeed the Tconv or mixed population of T cells used to generate the Tregs) can be isolated from an allogenic donor, preferably HLA matched, or from the subject diagnosed with a condition associated with the aberrant, unwanted or otherwise inappropriate immune response to a Smith protein. Preferably, the condition is SLE.

The T cells may also be generated from differentiation of induced pluripotent cells (iPSCs) or embryonic stem cells, preferably an embryonic stem cell line. The skilled person will be familiar with standard techniques for generating Treg cells from a stem cells, including an iPSC. Examples of these techniques are described in: Hague et al., (2012) J. Immunol., 189:2338-36; and Hague et al., (2019) JCI Insight, 4: pii 126471).

Further still, in the context of a mixed population of T cells, the skilled person will be familiar with standard techniques for isolating the subpopulation of the T cells which are CD4+CD25+ T cells (Treg cells). For example, CD4+CD25+ T cells (Treg cells) can be obtained from a biological sample from a subject by negative and positive immuno-selection and cell sorting.

In any method of the invention the Treg cells that have been cultured in the presence of a nucleic acid or vector can be transferred into the same subject from which cells were obtained. In other words, the cells used in a method of the invention can be an autologous cell, i.e., can be obtained from the subject in which the medical condition is treated or prevented. Alternatively, the cell can be allogenically transferred into another subject. Preferably, the cell is autologous to the subject in a method of treating or preventing a medical condition in the subject.

As used herein, the term “ex vivo” or “ex vivo therapy” refers to a therapy where cells are obtained from a patient or a suitable alternate source, such as, a suitable allogenic donor, and are modified, such that the modified cells can be used to treat a disease which will be improved by the therapeutic benefit produced by the modified cells. Treatment includes the administration or re-introduction of the modified cells into the patient. A benefit of ex vivo therapy is the ability to provide the patient the benefit of the treatment, without exposing the patient to undesired collateral effects from the treatment.

The term “administered” means administration of a therapeutically effective dose of the aforementioned composition including the respective cells to an individual. By “therapeutically effective amount” is meant a dose that produces the effects for which it is administered. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

An “enriched” or “purified” population of cells is an increase in the ratio of particular cells to other cells, for example, in comparison to the cells as found in a subject's body, or in comparison to the ratio prior to exposure to a peptide, nucleic acid or vector of the invention. In some embodiments, in an enriched or purified population of cells, the particular cells include at least 20%, 30%, 40%, 50%, 60%, 70%, 75%, 80%, 90%, 95% or 99% of the total cell population. A population of cells may be defined by one or more cell surface markers and/or properties.

Treg cells that express a binding protein as described herein can be administered to the subject by any method including, for example, injection, infusion, deposition, implantation, oral ingestion, or topical administration, or any combination thereof. Injections can be, e.g., intravenous, intramuscular, intradermal, subcutaneous or intraperitoneal, preferably intravenous. Single or multiple doses can be administered over a given time period, depending upon the condition, the severity thereof and the overall health of the subject, as can be determined by one skilled in the art without undue experimentation. The injections can be given at multiple locations.

Administration of the Treg cells can be alone or in combination with other therapeutic agents. Each dose can include about 10×10³ CD8+ Treg cells, 20×10³ cells, 50×10³ cells, 100×10³ cells, 200×10³ cells, 500×10³ cells, 1×10⁶ cells, 2×10⁶ cells, 20×10⁶ cells, 50×10⁶ cells, 100×10⁶ cells, 200×10⁶, 500×10⁶, 1×10⁹ cells, 2×10⁹ cells, 5×10⁹ cells, 10×10⁹ cells, and the like. Administration frequency can be, for example, once per week, twice per week, once every two weeks, once every three weeks, once every four weeks, once per month, once every two months, once every three months, once every four months, once every five months, once every six months, and so on. The total number of days where administration occurs can be one day, on 2 days, or on 3, 4, 5, 6, 7, 8, 9, 10, 11, 12, 13, 14, 15, 16, 17, 18, 19, or 20days, and so on. It is understood that any given administration might involve two or more injections on the same day. For administration, at least 20%, at least 30%, at least 40%, at least 50%, at least 60%, at least 70%, at least 80%, at least 90%, at least 95%, at least 99%, of the Treg cells that are administered exhibit at least one property of a Treg cell.

The Tregs described herein are useful for suppressing an aberrant immune response to an autoantigen, particularly to SmD, SmB/B′, Ro60 or MPO proteins. The Tregs or mixed population of Tregs can be administered in addition to or in place of any accepted protocols for treating the autoimmune disease characterised by an aberrant immune response to one or more of SmD, SmB/B′, Ro60 or MPO proteins.

In certain instances, the administration of immunosuppressive agents is decreased after administration of the Tregs. The dose of the immunosuppressive agent can be decreased by about 10%, 20%, 30%, 40%, 50%, 60%, 70%, 80%, 90%, or 100% after administration of the Tregs or the mixed population of Tregs. In some examples, the dose of the immunosuppressive agent is decreased by about 50% following treatment with Tregs or the mixed population of Tregs. In another example, the administration of immunosuppressive agents is ceased after administration of the Tregs or the mixed population of Tregs.

The Tregs described herein can be used in combination with other known agents and therapies. Administered “in combination,” as used herein, means that two (or more) different treatments are delivered to the subject during the course of the subject's affliction with the disorder (e.g., disease or condition), e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The Tregs described herein and the at least one additional therapeutic agent can be administered simultaneously, in the same or in separate compositions, or sequentially. For sequential administration, the Tregs can be administered first, and the additional agent can be administered second. Alternatively, the order of administration can be reversed, and the additional agent can be administered first, and the Tregs can be administered second. The T regs and/or other therapeutic agents, procedures, or modalities can be administered during periods of active disorder, or during a period of remission or less active disease. The Treg cell therapy can be administered before another treatment, concurrently with the treatment, post-treatment, or during remission of the disorder.

When administered in combination, the Tregs described herein and the additional agent (e.g., second or third agent), or all, can be administered in an amount or dose that is higher, lower, or the same as the amount or dosage of each agent used individually, e.g., as a monotherapy. In certain embodiments, the administered amount or dosage of the Tregs, the additional agent (e.g., second or third agent), or all, is lower (e.g., at least 20%, at least 30%, at least 40%, or at least 50%) than the amount or dosage of each agent used individually. In other embodiments, the amount or dosage of the Tregs, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect.

For example, the additional therapeutic agent(s) may include one or more immunosuppressive agents commonly given for treatment of the autoimmune disease (SLE, Sjogren's Syndrome, APA and other diseases described herein). The immunosuppressive agent(s) may be an agent that is given immediately after transplantation to prevent acute rejection (e.g., methylprednisolone, atgam, thymoglobulin, basiliximab, or alemtuzemab) or an immunosuppressive agent(s) used for maintenance (e.g., prednisone, a calcineurin inhibitor (e.g., cyclosporine or tacrolimus), mycophenolate mofetil, azathioprine, sirolimus or everolimus). Other immunosuppressive agents given after organ transplantation include CTLA-4 fusion proteins (e.g., belatacept or abatacept), corticosteroids (e.g., methylprednisolone, dexamethasone, or prednisolone), cytotoxic immunosuppressants (e.g., azathioprine, chlorambucil, cyclophosphamide, mercaptopurine, or methotrexate), immunosuppressant antibodies (e.g., antithymocyte globulins, basiliximab, or infliximab), sirolimus derivatives (e.g., everolimus or sirolimus), and anti-proliferative agents (e.g., mycophenolate mofetil, mycophenolate sodium, or azathioprine). Further immunosuppressants suitable for use the invention described herein are known to those of skill in the art, and the invention is not limited in this respect.

An effective amount of a therapeutic agent (e.g., a Treg, or a mixed population of Tregs, specific to a donor alloantigen or an autoantigen) described herein for treatment or prevention of an autoimmune disorder can be administered to a subject by standard methods. For example, the agent can be administered by any of a number of different routes including, e.g., intravenous, intraperitoneal, intramuscular, intradermal, subcutaneous, percutaneous injection, oral, transdermal (topical), transarterial, intratumoral, intranodal, intramedullar, or transmucosal.

In some embodiments, the agent (e.g., a Treg, or a mixed population of Tregs comprising Tregs specific to an autoantigen) can be administered (e.g., by injection or infusion) directly into affected tissue. In one embodiment, the compositions described herein are administered into a body cavity or body fluid (e.g., ascites, pleural fluid, peritoneal fluid, or cerebrospinal fluid). For example, the therapeutic agent (e.g., a Treg, or a mixed population of Tregs, specific to a donor alloantigen or an autoantigen) can be administered by injection or infusion, e.g., intramuscularly, subcutaneously, intraperitoneally, or intravenously. The most suitable route for administration in any given case will depend on the particular agent administered, the patient, the particular disease or condition being treated, pharmaceutical formulation methods, administration methods (e.g., administration time and administration route), the patient's age, body weight, sex, severity of the diseases being treated, the patient's diet, and the patient's excretion rate. The agent (e.g., a Treg, or a mixed population of Tregs, specific to a donor alloantigen or an autoantigen) can be encapsulated or injected, e.g., in a viscous form, for delivery to a chosen site. The agent can be provided in a matrix capable of delivering the agent to the chosen site. Matrices can provide slow release of the agent and provide proper presentation and appropriate environment for cellular infiltration. Matrices can be formed of materials presently in use for other implanted medical applications. The choice of matrix material is based on any one or more of: biocompatibility, biodegradability, mechanical properties, and cosmetic appearance and interface properties. One example is a collagen matrix.

The therapeutic agent (e.g., a Treg, or a mixed population of Tregs, specific to a donor alloantigen or an autoantigen) can be incorporated into pharmaceutical compositions suitable for administration to a subject, e.g., a human. Such compositions typically include the agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration.

The use of such media and agents for pharmaceutically active substances are known. Except insofar as any conventional media or agent is incompatible with the active compound, such media can be used in the described herein. Supplementary active compounds can also be incorporated into the compositions.

The term “unit dosage form” as is used herein refers to a dosage for suitable one administration. By way of example, a unit dosage form can be an amount of therapeutic disposed in a delivery device, e.g., a syringe or intravenous drip bag. For example, a unit dosage form is administered in a single administration. In another example, more than one unit dosage form can be administered simultaneously.

In some embodiments, the Tregs or mixed population of Tregs are administered as a monotherapy, i.e., another treatment for the condition is not concurrently administered to the subject. The Treg or mixed population of Tregs compositions can be administered once to the patient. If necessary, the Treg cell compositions can also be administered multiple times. The Tregs or mixed population of Tregs can be administered by using infusion techniques that are commonly known in immunotherapy (see, e.g., Rosenberg et al., New England Journal of Medicine. 319:1676 (1988)).

The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In other embodiments, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Nucleic Acids and Vectors

In another aspect, the present invention provides for the use of a nucleic acid molecule, construct or composition comprising one or more nucleic acid molecules encoding or complementary to a sequence encoding the binding proteins described herein, homologue or analogue thereof. The nucleic acid molecules may be used to produce a binding protein described herein, or used for cell therapy to treat a disease or condition described herein.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic or synthetic nucleic acid molecules. Exemplary vectors are those capable of autonomous replication (episomal vector) or expression of nucleic acid molecules to which they are linked (expression vectors).

Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In any aspect, a vector for use in accordance with the methods of the invention may comprise any one of more, or all, of the following:
an EF1α (alpha) promoter;
a 2A ribosome skipping sequence;
a woodchuck hepatitis virus post-transcriptional regulatory element (WPRE);
arrangement to translate TCR β-chain variable (Vβ or Vbeta) domain prior to the (TCR) α-chain variable (Vα or Valpha) domain; or
arrangement to translate TCR β-chain variable (Vβ or Vbeta) chain prior to the (TCR) α-chain variable (Vα or Valpha) chain.

Preferably, the vector is a lentiviral vector.

The term “operably-linked” refers to the association of two or more nucleic acid molecules on a single nucleic acid fragment so that the function of one is affected by the other. For example, a promoter is operably-linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a nucleic acid construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

The term “introduced” in the context of inserting a nucleic acid molecule into a cell, means “transfection”, or ‘transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein or other activity encoded by an exogenous nucleic acid molecule introduced into the host cell, even if the host cell encodes a homologous protein or activity.

As described herein, more than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous or exogenous nucleic acid molecules encoding desired TCR specific for a peptide described herein (e.g., TCR-α and TCR-β). When two or more exogenous nucleic acid molecules are introduced into a host cell, it is understood that the two or more exogenous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

The term “homologous” or “homolog” refers to a molecule or activity found in or derived from a host cell, species or strain. For example, a heterologous or exogenous nucleic acid molecule may be homologous to a native host cell gene, and may optionally have an altered expression level, a different sequence, an altered activity, or any combination thereof.

Host cells can be transformed to express the nucleic acids of the invention using conventional techniques such as calcium phosphate or calcium chloride co-precipitation, DEAE-dextran-mediated transfection or electroporation. Suitable methods for transforming the host cells may be found in Sambruck et al. (1989), and other laboratory texts. The nucleic acid sequence of the invention may also be chemically synthesised using standard techniques.

As used herein, the term “host” refers to a cell (e.g., Treg cell) or microorganism targeted for genetic modification with a heterologous or exogenous nucleic acid molecule to produce a polypeptide of interest (e.g., high or enhanced affinity TCR). In certain embodiments, a host cell may optionally already possess or be modified to include other genetic modifications that confer desired properties related or unrelated to biosynthesis of the heterologous or exogenous protein (e.g., inclusion of a detectable marker; deleted, altered or truncated endogenous TCR; increased co-stimulatory factor expression). In some embodiments, host cells are genetically modified to express a bind protein described herein.

### Conditions for Treatment

The present invention relates to methods for treating various autoimmune diseases, particularly autoimmune diseases characterised by an aberrant immune response (such as formation of autoantibodies), to one or more of SmB/B′, SmD, Ro60 and MPO proteins. The methods typically involve providing a population of Treg cells in an individual requiring treatment, wherein the Treg cells express a TCR binding protein as described herein.

The phrase “therapeutically effective amount” generally refers to an amount of a cell expressing a binding protein, or peptide of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein.

As used herein, “preventing” or “prevention” is intended to refer to at least the reduction of likelihood of the risk of (or susceptibility to) acquiring a disease or disorder (i.e., causing at least one of the clinical symptoms of the disease not to develop in an individual that may be exposed to or predisposed to the disease but does not yet experience or display symptoms of the disease). Biological and physiological parameters for identifying such patients are provided herein and are also well known by physicians.

In particularly preferred embodiments, the methods of the present invention can be to prevent or reduce the severity, or inhibit or minimise progression, of a flare-up or symptom of a disease or condition as described herein. As such, the methods of the present invention have utility as treatments as well as prophylaxes.

The terms “treatment” or “treating” of a subject includes the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an autoimmune disease as herein described (eg SLE, Sjogren's syndrome, vasculitis, microscopic polyangiitis or sclerosis) including any objective or subjective parameter such as abatement; remission; lessening of the rate of worsening; lessening severity of the condition; stabilization, diminishing of symptoms or making the condition more tolerable to the individual; slowing in the rate of degeneration or decline; making the final point of degeneration less debilitating; or improving a subject's physical or mental well-being.

It will also be understood that the methods described herein can be used in combination with existing standard of care treatments/therapies for the relevant autoimmune disease requiring treatment. The skilled person will be familiar with existing standard of care approaches to treatment of the autoimmune disease requiring treatment including but not limited to the use of steroids, anti-malarials (hydroxychloroquine, cholorquine), immunosuppressants (azathioprine, methotrexate, mycophenolate mofeti, mucophenolic acid, tacrolimus, voclosporin, ciclosporin), kinase inibitors (baricitinib, tofacitinib, upaticitinib) and biologics (belimumab, rituximab, anifrolumab, ustekinumab, obinotuzumab). The present invention includes combinations of existing standard of care approaches with the specific methods of the present invention.

A “subject” herein is preferably human subject. Although the invention finds application in humans, the invention is also useful for veterinary purposes. The invention is useful for domestic or farm animals such as cattle, sheep, horses and poultry; for companion animals such as cats and dogs; and for zoo animals. It will be understood that the terms “subject” and “individual” are interchangeable in relation to an individual requiring treatment according to the present invention.

### Systemic Lupus Erythematosus

The present invention includes methods for treating systemic lupus erythematosus, particularly when associated with an aberrant immune response to one or more of SmB/B′, SmD, Ro60 protein, or MPO protein.

Systemic lupus erythematosus (SLE) is a multi-system autoimmune disease. At least 5 million people worldwide have SLE; 90% of those diagnosed are female and most develop the disease between the ages of 15-44. In Australia, SLE is diagnosed in ˜1 in 1000 people and is more prevalent and severe in Indigenous Australians and Asian Australians. SLE patients suffer chronic immune-mediated inflammatory damage in the brain, kidneys, heart, lungs, joints, skin, and other organs, resulting in a marked loss of life expectancy, exemplified by a standardized mortality ratio above 3. In a British cohort, the average age of death of the 14% of patients who died during follow-up was only 52 years. Most often the clinical course is characterised by episodic flares, which are associated with accrual of irreversible organ damage and thereby mortality.

Other forms of lupus include discoid, drug-induced and neonatal lupus. Of these, systemic lupus erythematosus (also known as SLE) is the most common and serious form. A more thorough categorization of lupus includes the following types: acute cutaneous lupus erythematosus, subacute cutaneous lupus erythematosus, discoid lupus erythematosus (chronic cutaneous), childhood discoid lupus erythematosus, generalized discoid lupus erythematosus, localized discoid lupus erythematosus, chilblain lupus erythematosus (Hutchinson), lupus erythematosus-lichen planus overlap syndrome, lupus erythematosus panniculitis (lupus erythematosus profundus), tumid lupus erythematosus, verrucous lupus erythematosus (hypertrophic lupus erythematosus), cutaneous lupus mucinosis, complement deficiency syndromes, drug-induced lupus erythematosus, neonatal lupus erythematosus, systemic lupus erythematosus.

Cutaneous lupus erythematosus (CLE) is seen in the majority of SLE cases and is most often observed in skin exposed to the sun, appearing as a variety of severe and in some cases disfiguring skin rashes. Lupus may also manifest as a purely cutaneous form, also known as incomplete lupus erythematosus. While all the factors leading to the development of SLE, and its pattern of intermittent flares, are not known, it is clear that sunlight exposure is important in systemic as well as cutaneous disease exacerbation.

Of the symptoms common to those diagnosed with lupus, almost all patients have joint pain and/or swelling (i.e., arthritis). Frequently affected joints are the fingers, hands, wrists, and knees. Other common symptoms include: pleuritic chest pain, oral and nasal ulcers, fatigue, fever with no other cause, general discomfort, uneasiness, or ill feeling (malaise), hair loss, sensitivity to sunlight, skin rash—a “butterfly” rash in about half people with SLE and also scarring “discoid” lesions, and swollen lymph nodes. The skilled person will be familiar with various other important manifestations of lupus, including but not limited to: nephritis, CNS involvement, haematological involvement, gastrointestinal involvement, and vasculitis.

As used herein, photosensitivity or abnormal light sensitivity in an individual with CLE or SLE includes skin rashes that result of unusual reaction to sunlight. Beyond skin rashes that can develop, exposure to the sun can cause those living with lupus to experience increased disease activity with symptoms such as joint pains, weakness, fatigue and fever. Two-thirds of people with lupus have increased sensitivity to ultraviolet rays, either from sunlight or from artificial inside light, such as fluorescent light—or both.

### Sjogren's Syndrome

The present invention includes methods for treating Sjogren's Syndrome particularly when associated with an aberrant immune response to one or more of SmB/B′, SmD, Ro60 protein, or MPO protein.

Sjogren's Syndrome, and in particular, Primary Sjogren's Syndrome (pSS) is an autoimmune disorder that is estimated to afflict between 0.5% to 1% of the general population, of whom nine out of ten patients are women. The majority of women with pSS are characterized by mild to moderate disease which manifests as fatigue, joint pain, and ocular and/or oral dryness. The disease is characterized by the lymphocytic infiltration of salivary and lacrimal glands with subsequent inflammation, damage and loss of function of the glands causing dry eyes and dry mouth. Involvement of major organ systems including lung, kidney, and liver are common systemic manifestations of pSS. At a biochemical level, pSS is associated with increased immunoglobulin levels and the production of anti-nuclear antibodies against ribonucleoprotein complexes such as SSA/Ro and SSB/La.

Fatigue is one of the most common extraglandular symptoms of Sjogren's syndrome and is defined by enduring generalized tiredness. An estimated 70% of pSS patients suffer from profound fatigue, which is reported to have a negative impact on quality of life. Serologically, approximately 80% of these patients have anti-Ro/SSA autoantibodies which bind to autoantigens containing small non-coding RNA molecules. Fatigue can be characterized in terms of intensity, duration, and effects on daily function. Notably, in the primary care setting fatigue is strongly associated with depression. Thus, there exists a need for a means to improve fatigue in patients with autoimmune diseases such as Sjogren's syndrome.

As used herein, the terms “primary Sjogren's syndrome (pSS),” “Sjogren's syndrome,” “Sjogren's disease,” and “Sjogren's” can be used interchangeably.

In any embodiment, treatment of Sjogren's syndrome may comprise reducing or decreasing fatigue in the subject requiring treatment. Various patient reported outcome (PRO) instruments have been used and validated in the measurement of fatigue in subjects with chronic diseases. Such PROs are known in the art and can be used to assess the efficacy of treatment with a population of T reg cells as described herein. The European League Against Rheumatism (EULAR) Sjogren's Syndrome (SS) Patient Reported Index (ESSPRI) was developed to assess the symptoms of patients with primary Sjogren's syndrome (Seror et al., Ann. Rheum. Dis. 2011;70:968-972). ESSPRI was developed as a global score to measure all important and disabling symptoms of primary Sjogren's syndrome: dryness, limb pain, and fatigue. ESSPRI has been shown to be sufficient to measure each of the symptoms without loss of content validity and the score is easy to calculate. The ESSPRI is a patient-administered questionnaire that assess the symptoms of patients with primary Sjogren's syndrome. The questionnaire includes three scales, one for each of the following symptoms: (1) dryness, (2) limb pain, and (3) fatigue. Each component of the ESSPRI is measured with a single 0-10 numerical scale and the global ESSPRI score is the mean of the three scales: (dryness+limb pain+fatigue)/3. A decrease of at least one point in the ESSPRI score is clinically meaningful.

The Functional Assessment of Chronic HIness Therapy Fatigue scale (FACIT-Fatigue) is used to assess an individual's level of fatigue during their usual daily activities over the past week. The FACIT-Fatigue questionnaire and Scoring & Interpretation Materials are available from FACIT.org (Elmhurst, III., USA). The FACIT-Fatigue questionnaire provides an array of generic and targeted measures. The FACIT fatigue scale has many benefits including high internal validity, high test-retest reliability, reliability and sensitivity to change in patients with a variety of chronic health conditions, ease of use, and use in a variety of settings. (K. F. Tennant, Try This: best Practices in Nursing Care to Older Adults, Issue 30, 2012; Chandran et al., Ann. Rheum. Dis. 2007; 66:936-939). The FACIT-Fatigue is a 13-item questionnaire originally developed to measure fatigue in patients with cancer and is now use in patients with Sjogren's disease to measure fatigue. The patient is asked to answer 13 questions scored from 0to 4 (O=not at all, 1=a little bit, 2=somewhat, 3=quite a bit, 4=very much). The fatigue scale has 13 items, with 52 as the highest possible score. A higher score in the fatigue scale corresponds to a lower level of fatigue and indicates better quality of life. To calculate the FACIT-fatigue score, the response scores on negatively phrased questions are reversed and then the 13 item responses are added. Eleven items with responses have their scores reversed (item score=4-response, if the response is not missing), and two items (items 7-8) have their responses unchanged. All items are added so that higher scores correspond to less fatigue. In cases where individual questions are skipped, scores are prorated using the average of other answers in the scale. FACIT-Fatigue=13* [sum(reversed items)+sum(items 7-8)]/number of answered items.

The Profile of Fatigue (ProF) was developed to establish an assessment tool that was effective in characterizing fatigue associated with primary Sjogren's syndrome. The ProF has been shown to be a reliable and valid instrument for measuring the severity of fatigue and general discomfort in patients with primary Sjogren's syndrome. The ProF is a 16 item self-administered questionnaire divided into two domains, one for somatic fatigue and one for mental fatigue. The somatic fatigue domain includes 12items divided into four facets: (a) need rest (four items), (b) poor starting (three items), (c) low stamina (three items), and (d) weak muscles (two items). The mental fatigue domain includes 4 items is divided into two facets: (a) poor concentration (two items) and (b) poor memory (two items). Patient's score each item on a scale of 0-7 (0=‘no problem at all’ and 7=“as bad as imaginable’) based on how the patient felt at their worst over the past two weeks. The score for each facet can be obtained by adding up the item scores within each facet and dividing the sum by the number of items in each facet. The score for each domain (e.g., somatic, mental) can be obtained by adding up the facet scores within each domain and dividing the sum by the number of facets within each domain. Higher scores indicates greater fatigue. (Bowman et al., Rheumatology, 2004; 43:758-764; Strombeck et al., Scand. J. Rheumatol. 2005;34:455-459; Segal et al., Arthritis Rheum. 2008 Dec. 15; 59 (12): 1780-1787).

In some embodiments, the patient's condition is evaluated by measuring fatigue in the patient by one or more patient reported indices (e.g., ESSPRI, PROF, FACIT) as compared to the level of fatigue in the patient prior to treatment or relative to the levels of fatigue in a similarly afflicted untreated or control patient.

For example, a human subject in need of treatment is selected or identified (e.g., a patient who fulfills the American College of Rheumatology criteria for SLE, or a patient who fulfills the American-European Consensus Sjogren's Classification Criteria). The subject can be in need of, e.g., reducing a cause or symptom of SLE or Sjogren's syndrome, such as fatigue. The identification of the subject can occur in a clinical setting, or elsewhere, e.g., in the subject's home through the subject's own use of a self-testing kit. The patient's condition is evaluated at baseline (day 1) and after a period of time following the first dose, e.g., day 8, day 15, day 29, day 43, day 57, day 71, day 85,day 99 or at the end of the study, e.g., by ESSPRI index, PROF, and/or the FACIT fatigue scale. Other relevant criteria can also be measured. The number and strength of doses are adjusted according to the subject's needs. After treatment, an improvement in one or more of the following outcomes can be noted: (1) an improvement in the ESSPRI index relative to the ESSPRI index prior to treatment, or relative to a similarly afflicted but untreated/control subject, (2) an improvement in the PROF relative to the PROF prior to treatment, or relative to a similarly afflicted but untreated/control subject, (3) an improvement can be noted in the FACIT fatigue scale relative to the FACIT fatigue scale prior to treatment, or relative to a similarly afflicted but untreated/control subject. In some embodiments, an improvement in the ESSPRI index is a clinically meaningful improvement. A clinically meaningful improvement in the ESSPRI index is a decrease of at least one point in the ESSPRI score.

Various neuropsychological assays known in the art can also be used to assess the efficacy of the methods of treatment disclosed herein, including to assess improvement in Sjogren's Syndrome Associated Cognitive Function. For example the Digit Symbol Substitution Test (DSST) provides a valid and sensitive test to measure cognitive dysfunction that is impacted by many domains. The DSST is sensitive to both the presence of cognitive dysfunction as well as a change in cognitive function across a range of clinical populations, including patients with Sjogren's Syndrome. This neuropsychological test is widely used, highly validated, and extremely sensitive test reading out on executive function related inputs.

DSST is a time limited paper-and-pencil cognitive test that is given on a single sheet of paper. The test requires a patient to match symbols to numbers according to a key at the top of the paper. The patient copies the symbol into spaces below a row of numbers and the number of correct symbols within the allowed time (e.g., 90 or 120 seconds) is calculated. The test provides data on the accuracy and rate of performing the task. A patients performance on the DSST correlates with real-world functional outcomes, such as the ability to accomplish everyday tasks, and recovery from functional disability in a range of psychiatric conditions. The DSST test can be used to assess attention and/or focus in the patient.

DSST is a polyfactorial test that measures a range of cognitive operations and provides a practical and effective method to monitor cognitive function over time. To perform well on the DSST the patient must have intact motor speed, attention and visuoperceptual functions, including scanning and the ability to write or draw (i.e., basic mental dexterity). DSST offers high sensitivity to detect cognitive impairment and has many benefits including brevity, reliability, sensitivity to change, and minimal impact on language, culture and education on test performance. (Jaeger, J., Journal of Clinical Psycopharmacology, 38(5), 513-518, October 2018).

### Systemic Sclerosis

The present invention includes methods for treating systemic sclerosis particularly when associated with an aberrant immune response to one or more of SmB/B′, SmD, Ro60 protein, or MPO protein.

Systemic sclerosis (Ssc, also known as scleroderma) is a non-inherited, non-infectious, multisystem autoimmune disease that can result in progressive fibrosis of the skin and/or internal organs. The condition is characterized by excessive collagen deposition. One form of the condition known as CREST syndrome (also referred to as limited scleroderma) can result in the following features: calcinosis, Raynaud's phenomenon, esophageal dysmotility, sclerodactyly, and telangiectasia.

SSc has a broad variety of symptoms triggered by excessive deposition of extracellular matrix in the dermis resulting in skin fibrosis. In later stages SSc is characterized by progressive tissue fibrosis affecting other internal organs as the gut, the lung or the kidneys. Therefore scleroderma is the hallmark of the disease comprising also e.g. lung fibrosis, renal fibrosis, fibrosis of the heart, the gut or the blood vessels. It is suggested that inflammation, autoimmune disorders or vascular damage activates fibroblasts. Fibroproliferation is accompanied by excessive extracellular matrix production, dominated by Collagen type I resulting in progressive tissue fibrosis which can cause end organ failure and lead to high morbidity and mortality in patients with end-stage SSc.

The present invention contemplates the treatment of Systemic Sclerosis (SSc), diffuse Systemic Sclerosis (dSSc), limited Systemic Sclerosis (ISSc), overlap type of Systemic Sclerosis, undifferentiated type of Systemic Sclerosis, Systemic Sclerosis sine scleroderma, skin fibrosis, scleroderma, nephrogenic fibrosing dermopathy (NFD), and/or keloid formation.

In some embodiments, an individual requiring treatment for scleroderma is assessed for inflammation, fibrosis, vasculopathy, and/or autoimmunity. In some embodiments, an individual is assessed for at least one of the following: skin thickening, skin thickening proximal to metacarpophalangeal (MCP) joints, skin thickening of the fingers, puffy fingers, sclerodactyly, fingertip lesions, digital tip ulcers, pitting scars, telangiectasia, abnormal nailfold capillaries, calcinosis, dilated esophagus, scleroderma renal crisis, interstitial lung disease, pulmonary arterial hypertension and/or interstitial lung disease, Raynaud's phenomenon, related antibodies (e.g., an anti-centromere antibody, anti-Scl-70 antibody/anti-topoisomerase antibody, anti-fibrillarin autoantibody, anti-RNA polymerase III autoantibody, anti-Th/To, PM-ScI, anti-Ro, anti-UI-ribonucleoprotein, etc.), and collagen mRNA levels in the skin. In some embodiments, the scleroderma is localized to the skin. In some embodiments, the scleroderma involves at least one organ other than the skin. In some embodiments, the scleroderma is referred to as systemic sclerosis. In some embodiments, the scleroderma is CREST syndrome.

In some embodiments, the condition is assessed using a plasma sample. In some embodiments, the condition is assessed using a blood sample. In some embodiments, the condition is assessed using a skin biopsy sample. In some embodiments, the condition is assessed using a fibrotic biomarker. In some embodiments, the condition is assessed using detection of a condition-specific autoantibody.

In some embodiments, the condition is assessed using the modified Rodnan skin score (MRSS). In some embodiments, the MRSS scores for each body site are as follows: 0=no skin involvement; I=mild thickening; 2=moderate thickening; and 3=severe thickening. In some embodiments, the MRSS at a body site is at least 2. In some embodiments, the individual has moderate skin thickening. In some embodiments, skin thickness is measured in 17 different body sites. In some embodiments, the individual has a total body MRSS score of at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51.

In some embodiments, the condition is assessed using palpation. In some embodiments, the condition is assessed using palpation on 17 different body areas. In some embodiments, the body areas include at least one of the following: fingers, hands, forearms, arms, feet, legs, and thighs (bilaterally) and face, chest, and abdomen (singly).

In some embodiments, the condition is assessed using the localized scleroderma cutaneous assessment tool (LoSCAT). In some embodiments, the LoSCAT assesses 18 cutaneous anatomic sites.

In some embodiments, the LoSCAT captures disease activity (mLoSSI) and damage (LoSDI) parameters. In some embodiments, the LoSCAT takes into consideration cutaneous lesions resulting from localized scleroderma diseases during the inactive stage of the disease. In some embodiments, scores for each site are based on the most severe score for each parameter. In some embodiments, skin changes are compared with the contralateral or ipsilateral skin area to minimize inter-subject variability.

In some embodiments, the Leroy and Medsger criteria is used. In some embodiments, the 1988 Leroy and Medsger criteria is used. In some embodiments, the 2001 Leroy and Medsger criteria is used. In some embodiments, the condition is assessed using the American College of Rheumatology (ACR) criteria. In some embodiments, the condition is assessed using the European League Against Rheumatism (EULAR) criteria. In some embodiments, the 2013 ACR/EULAR diagnostic criteria is used. In some embodiments, an individual is diagnosed with systemic sclerosis if they have a total score of at least 9 using the 2013 ACR/EULAR diagnostic criteria.

In some embodiments, treatment is assessed using the change in a score described herein. In some embodiments, treatment is assessed using the percent change in a measurement described herein. In some embodiments, treatment is assessed using changes in the tele-thermographic profile of cutaneous lesions following treatment. In some embodiments, treatment is assessed using changes in the ultrasound profile of target cutaneous lesion following treatment.

In some embodiments, the condition is assessed using a computed tomography (CT) scan of the lungs. In some embodiments, the condition is assessed by measuring kidney function. In some embodiments, the condition is assessed by measuring blood levels of creatinine. In some embodiments, the condition is assessed using a pulmonary function test. In some embodiments, the condition is assessed using a forced vital capacity (FVC) measure of lung capacity. In some embodiments, the condition is assessed using a diffusing capacity (DLCO) measure of oxygen exchange in the alveoli.

In some embodiments, the condition is assessed using the Dermatology Life Quality Index (DLQI). In some embodiments, the condition is assessed using the Health Assessment Questionnaire-Disability Index (HAQ-DI). In some embodiments, the condition is assessed using the physician global assessment (PGA).

### Inflammatory Myositis

The present invention includes methods for treating inflammatory myositis. particularly when associated with an aberrant immune response to one or more of SmB/B′, SmD, Ro60 protein, or MPO protein.

Inflammatory myositis is a systemic autoimmune disease characterised by inflammation of the muscles. Examples of inflammatory myositis conditions that can be treated as described herein include, without limitation, polymyositis, dermatomyositis, inclusion body myositis, and juvenile myositis.

Any appropriate method can be used to determine whether or not a subject has an inflammatory myositis condition. For example, a subject (e.g., human) can be identified as having an inflammatory myositis condition using standard diagnostic techniques. In some cases, a tissue biopsy can be collected and analyzed to determine whether or not the subject has an inflammatory myositis condition.

Any appropriate method can be used to determine whether or not the severity of an inflammatory myositis condition is reduced following treatment according to the methods of the present invention. For example, the severity of an inflammatory myositis condition can be assessed by visual inspection, diagnostic techniques, or patient questioning.

### Autoimmune Vasculitis, Including Microscopic Polyangiitis

The present invention includes methods for treating autoimmune vasculitis, including microscopic polyangiitis, particularly when associated with an aberrant immune response to MPO protein, or to one or more of SmB/B′, SmD, or Ro60 protein.

Autoimmune vasculitis, is an autoimmune disease that causes inflammation and narrowing of blood vessels (arteries, veins and capillaries). In severe cases, the condition can cause organ damage or death. Types of vasculitis are grouped according to the size of the blood vessels affected. Most types of vasculitis are rare and include: large vessel (Polymyalgia rheumatic, Takayasu's arteritis, temporal arteritis (giant cell arteritis)), medium vessel (Buerger's disease, cutaneous vasculitis, Kawasaki disease, polyarteritis nodosa), small vessel (Behçet's syndrome, Churg-Strauss syndrome, cutaneous vasculitis, Henoch-Schönlein microscopic polyangiitis, granulomatosis with polyangiitis, Golfer's vasculitis, cryoglobulinemia). Vasculitis symptoms may occur once or several times over several years. The disease affects people of all ages, races and gender.

Signs and symptoms of vasculitis vary and can range from mild to life-threatening. They depend on the type of vasculitis, the organs involved and how severe the condition is. Some people may have few signs and symptoms while other people may become very sick. Sometimes symptoms develop slowly, over months, while at other times, the signs and symptoms start quickly, over days or weeks.

Common symptoms include: fever, loss of appetite, weight loss, fatigue, general aches and pains. Vasculitis can affect various organs and body systems, causing a range of signs and symptoms including: skin (purple or red spots or bumps; clusters of small dots, splotches, bruises, or hives; itching), joints (aching or arthritis in one or more joints), lungs (shortness of breath; coughing up blood), gastrointestinal tract (sores in the mouth; stomach pain; in severe cases, blockage of blood flow to the intestines that can cause weakening or rupture of intestines), sinuses, nose, throat and ears (sinus or chronic middle ear infections; sores in the nose; in some cases, hearing loss), eyes (red, itchy, burning eyes; light sensitivity; blurred vision; rarely, blindness), brain (headaches; problems thinking clearly; changes in mental function; stroke-like symptoms, such as muscle weakness and paralysis), nerves (numbness, tingling and weakness in various parts of the body; loss of feeling or strength in hands and feet; shooting pains in arms and legs). In severe cases, vasculitis can cause blockages in blood vessels, possibly leading to aneurysm.

Microscopic polyangiitis is a necrotizing vasculitis which involves the small-caliber vessels and may be responsible for glomerula and lung capillary damage responsible for a pneumorenal syndrome, in addition to the systematic manifestations in connection with vasculitis.

Anti-neutrophil cytoplasmic antibodies (ANCAs) are autoantibodies formed against antigens in the cytoplasm of neutrophil granulocytes and monocytes. These are associated with a number of autoimmune disorders, particularly systemic vasculitis and ANCA-associated vasculitides (AAV). ANCA can be divided into four patterns when visualised by IF; cytoplasmic ANCA (c-ANCA), C-ANCA (atypical), perinuclear ANCA (p-ANCA) and atypical ANCA (a-ANCA), also known as x-ANCA. c-ANCA shows cytoplasmic granular fluorescence with central interlobular accentuation. C-ANCA (atypical) shows cytoplasmic staining that is usually uniform and has no interlobular accentuation. p-ANCA has three subtypes, classical p-ANCA, p-ANCA without nuclear extension and granulocyte specific-antinuclear antibody (GS-ANA). Classical p-ANCA shows perinuclear staining with nuclear extension, p-ANCA without nuclear extension has perinuclear staining without nuclear extension and GS-ANA shows nuclear staining on granulocytes only, a-ANCA often shows combinations of both cytoplasmic and perinuclear staining.

p-ANCA antigens include myeloperoxidase (MPO). Currently, the diagnosis of ANCA-positive vasculitis is still based on a biopsy, whether it is a skin biopsy, a renal biopsy, a neuromuscular biopsy, or the like. ANCAs constitute an important aid to the diagnosis of systemic vasculitis. Anti-myeloperoxidase (MPO) ANCAs are thus present in 60 to 75% of patients suffering from microscopic polyangiitis and 38% of patients suffering from Churg-Strauss syndrome.

In preferred embodiments of the invention, the treatment of autoimmune vasculitis comprises the treatment of a patient that has anti-MPO ANCAs. Examples of anti-MPO ANCA positive vasculitis includes microscopic angiitis (MPA), eosinophilic granulomatosis with polyangiitis (Churg Strauss syndrome) and in rare instances, Granulomatosis with polyangiitis (Wegener granulomatosis).

### EXAMPLES

### Example 1: Identification of Promiscuous TCR

The inventors co-cultured T cells from healthy individuals with 6 different self-peptides derived from 3 different known autoantigens (2× Smith; 2× Ro60; and 2× MPO). In all 6 cultures, the most reactive T cell clone (i.e. TCR) was found to be the same. In more detail:

### Methods

Approximately 50 ml whole blood was obtained via venipuncture into EDTA-coated Vacutainer tubes (BD Biosciences). Mononuclear cells (MNCs) were isolated using density centrifugation with Lymphoprep in 50 ml SepMate tubes according to the manufacturer's protocol (StemCell Technologies). Monocyte isolation was then performed on the MNCs using the EasySep Human Monocyte Isolation Kit (StemCell Technologies).

Isolated monocytes were stained with the proliferation dye CellTrace Far Red (Life Technologies), counted, and resuspended in ImmunoCult DC Differentiation Medium (StemCell Technologies) at 10⁶ cells/ml. Cells were plated in a 96-well polystyrene flat bottom plate (In-Vitro Technologies) at 10⁵ cells per well. A medium change was performed on day 3 of culture, and dendritic cell maturation supplement (Stemcell Technologies) was added on day 5 of culture according to the manufacturer's instructions.

On day 7 of monocyte culture, another 50 ml whole blood was obtained via venipuncture from the same healthy individuals expressing both HLA-DR3 and HLA-DR4.5. CD4+ T cells were isolated using the RosetteSep Human CD4+ T Cell Enrichment Kit (Stemcell Technologies). Resulting CD4+ T cells were labelled with CellTrace Violet (ThermoFisher Scientific), resuspended in warm supplemented RPMI media (10% human male AB serum, 2% penicillin/streptomycin, 4 mM L-glutamine and 50 μM 2-mercaptoethanol) and transferred into wells containing dendritic cells at a ratio of 1 CD4+ T cell to 1 dendritic cell.

Co-cultures were supplemented with 100 μg/ml peptide and 80 units/ml IL-2(StemCell Technologies).

Peptides used were:

SmD1:78-92, SmB/B′:7-21 (restricted to HLA-DR3 (DRA1*01:01+DRB1*03:01), Ro60:225-239, Ro60:369-383 (restricted to HLA-DR3 (DRA1*01:01+DRB1*03:01); MPO:453-467 and MPO:724-738 (restricted to HLA-DR4.5 (DRA1*01:01+DRB1*04:05). Sequences of the peptides tested are provided in Table 1 herein.

Cells were harvested on day 6 of culture and surface stained with BUV496labelled anti-human CD4 (BD Biosciences), APC-H7 labelled anti-human CD8 (BD Biosciences), BV711 labelled anti-human CD69 (BD Biosciences), AF488 labelled anti-human HLA-DR (Biolegend) and propidium iodide (Sigma).

Post-staining, cells were filtered through a 20 μm filter into sterile polypropylene tubes in ice-cold sterile MACS buffer then CD4+CTVlo cells were sorted using the BD Biosciences FACS Aria cell sorter. Sorted cells were then sent for 10' single cell TCR sequencing.

Healthy individual full HLA-type: HLA-A*11:01/33:03; HLA-B*58:01; HLA-C*03:02; HLA-DRB1*03:01/04:05; HLA-DRB3*02:02; HLA-DRB4*01:03; HLA-DPB1*04:01/05:01; HLA-DPA1*01:03/02:02; DQB1*02:01/04:01; DQA1*03:03/05:01.

The cultures were conducted on different days for different peptides (i.e. Sm peptides, Ro60 peptides and MPO peptide).

### Results

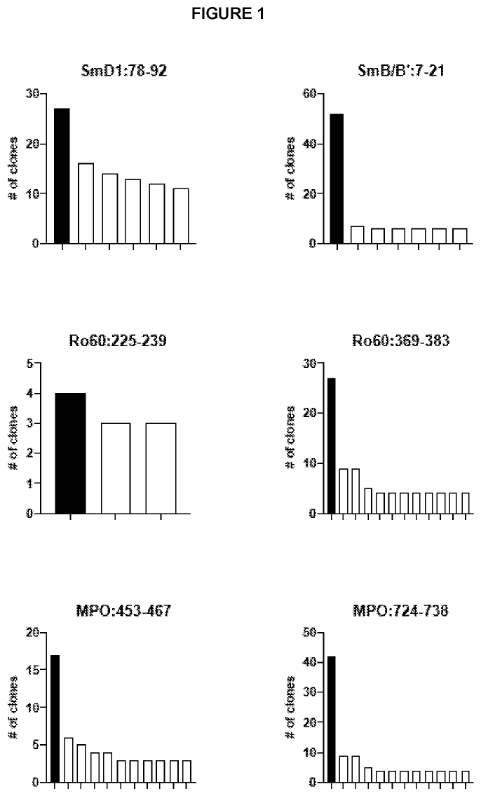

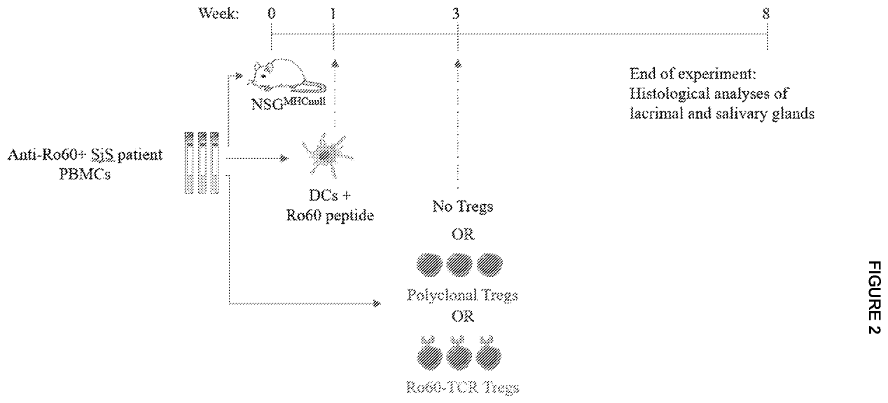

Single cell TCR sequencing revealed that the same Treg-derived TCR (as defined in Table 1 herein) was the dominant clonotype in all subjects (see FIG. 1). The results indicate that a single TCR recognizes 6 different self-peptide-MHC conformations and dominantly expands.

### Example 2

To demonstrate the promiscuity of the TCR, a Jurkat T cell line is transduced with TCR and stimulated with Sm peptides, Ro60 peptides and MPO peptides individually in the presence of DR3/DR4.5 expressing B-LCLs as antigen presenting cells. A positive response is determined on the transduced Jurkat T cell by measuring the upregulation of the T cell activation marker CD69, IL-2 production in the supernatant and/or proliferation.

To determine the efficacy of the TCR identified in Example 1 at suppressing anti-Ro60 specific pro-inflammatory responses, the effect of Tregs expressing the TCR on the expansion of pro-inflammatory Ro60-specific T conventional cells (Tconv) is measured using a proliferation assay. The results are expected to show that in the presence of Ro60-Tregs, the numbers of Tregs to Ro60-specific Tconv is markedly increased. This result means that the Ro60-Tregs potently suppresses the expansion of Ro60-specific Tconv cells.

Next, the inventors cytokine production is measured and the results are expected to show that in the presence of Ro60-Tregs an anti-inflammatory response, i.e. high IL-10, low IFN-g and IL-17A, is dominant (like in healthy individuals), whereas without Tregs or with only pTregs, a pro-inflammatory response is dominant, i.e. low IL-10,high IFN-γ and IL-17A (as is expected in autoimmune disease patients). These data will demonstrate that Ro60-Tregs have the capability to reset the aberrant immune response and restore tolerance to the targeted autoepitope.

### Example 3: HLA-DR3 Restricted Ro60-Tregs Halt the Progression of Sjogren's Syndrome

To demonstrate the efficacy of Ro60-Tregs at halting disease progression, the inventors will devise a new humanised model of Sjogren's Syndrome based on a similar published model (Young N A et al, Clin Immunol. 2015 January; 156(1): 1-8).

In this model, the adoptive transfer of PBMCs from patients with anti-Ro60 antibodies into immunocompromised NSG-MHC^{null} mice will lead to tissue injury in the lacrimal and salivary glands measured histologically by the infiltration of human T cells and loss of normal tissue structural integrity. At the onset of disease, mice are given either no Tregs, polyclonal Tregs (pTregs) or Ro60-Tregs. FIG. 2 provides a schematic of the experimental protocol.

Mice that received no Tregs or pTregs will develop dacryoadenitis and sialoadenitis that progress to destruction of lacrimal and salivary glands, however, the mice treated with Ro60-Tregs will display only minimal disease.

To demonstrate that the disease seen in this model is mediated by an anti-Ro60immune response, monocyte derived DCs isolated from Sjogren's Syndrome patients will be pulsed with Ro60 peptide and injected into mice 7 days after PBMC transfer. In this way, the anti-Ro60 specific T cells will be preferentially expanded and accelerate anti-Ro60 T cell mediated disease.

### Example 4: Upregulated Treg Genes Associated with Antigen-Specific TCR Activation

Method: CD4+ T cells from a HLA-DRB1*03:01/DRB1*04:05 healthy donor were co-cultured with monocyte derived dendritic cells (DCs) in the presence of the following individual autoepitopes: SmD1:78-92, SmB/B′:7-21, Ro60:369-383, MPO:453-467, MPO:724-738. Proliferating T cells were subjected to single cell sequencing analysis (10× Genomics). To determine if clonal expansion of the promiscuous self-TCR is linked with TCR activation, the inventors analysed the single cell transcriptome data comparing the gene expression profile with the other T cells and identified genes associated with TCR activation. In addition, the inventors also identified genes associated with Treg activation because the promiscuous self-TCR identified is derived from a Treg.

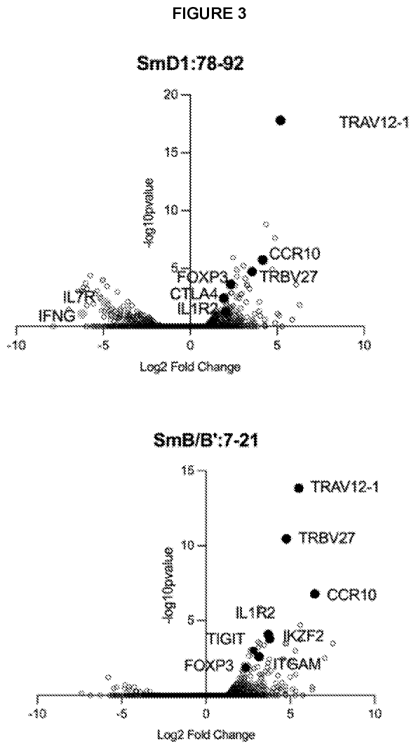

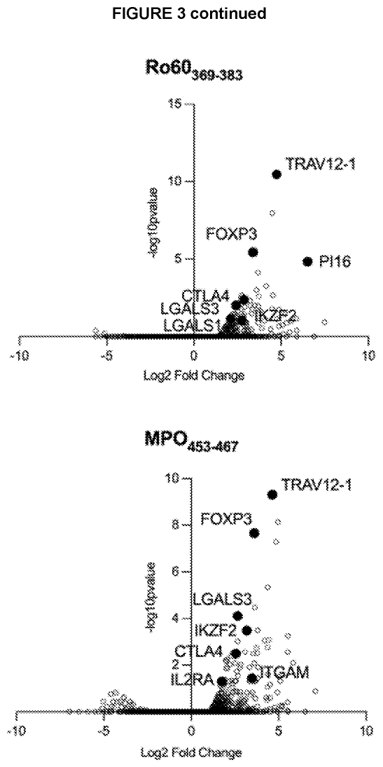

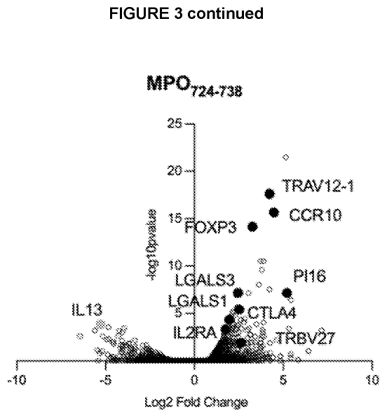

Result: As shown in FIG. 3, the upregulated genes associated with TCR activation were FOXP3, CTLA4, IL1R2, CCR10, TIGIT, IKZF2, ITGAM, LGALS1, LGALS3, PI16, IL2RA. Downregulated genes associated with Treg activation were IL13, IFNG, IL7R.

Conclusion: These data demonstrate that each epitope can induce promiscuous self-TCR engagement and Treg activation.

### Example 5: Expression of the Promiscuous Self-TCR on Human Tregs

Method: To study the advantageous immunosuppressive effects of adding the promiscuous self-TCR onto human Tregs, the inventors developed a lentiviral transduction process that enables expression of the promiscuous self-TCR on the surface of human Tregs. Firstly, the TCR sequences of the promiscuous self-TCR were cloned into a lentiviral vector and lentiviral stocks were obtained. Then, human Tregs were isolated from PBMCs by magnetic isolation and flow sorting, expanded in the presence of anti-CD2, anti-CD3, anti-CD28 and IL-2, then transduced with lentivirus at an MOI of 30. Transduction efficiency was measured by flow cytometry. GFP expression reflects the transduction efficiency and Vβ14 expression reflects the percentage of Tregs expressing the promiscuous self-TCR on the surface of the cell.

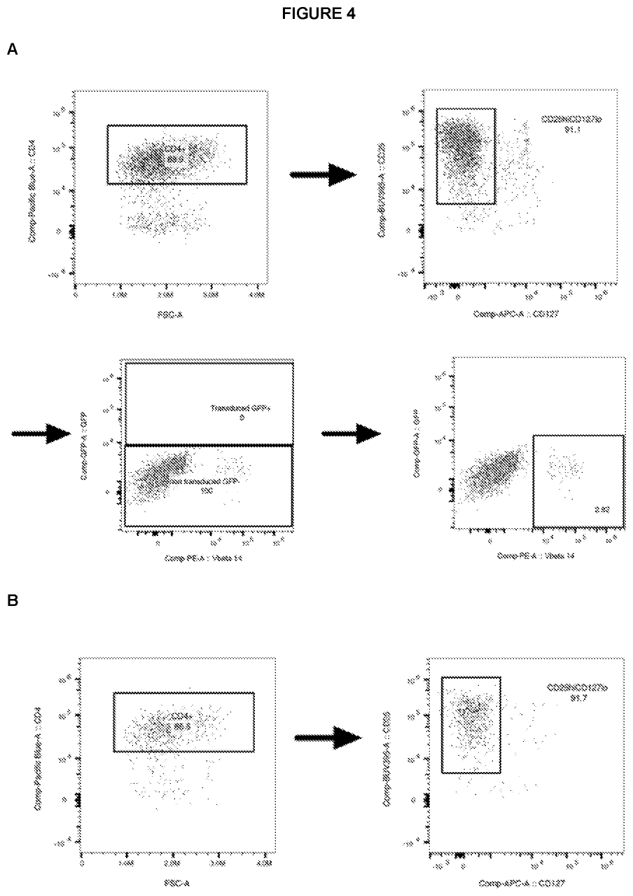

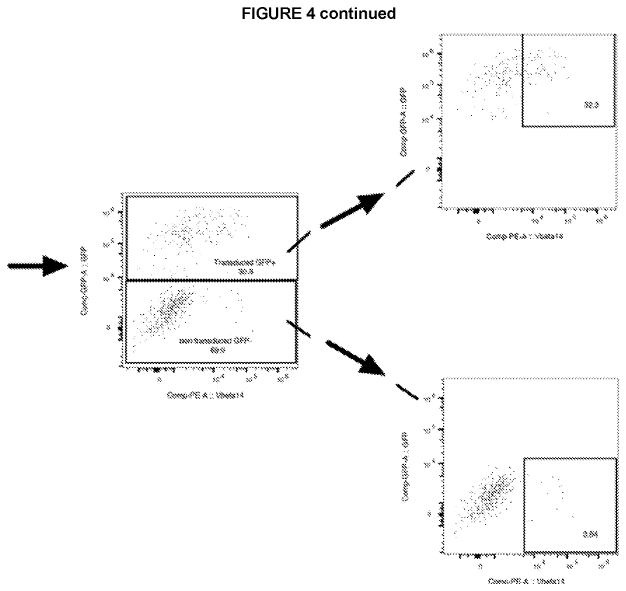

Result: As shown in FIG. 4, transduction efficiency of the Treg transduction protocol was 30.8%. Of the transduced GFP+ Tregs, 32.3% were positive for Vβ14. In non-transduced GFP-cells the endogenous Vβ14 expression was 3.84%.

Conclusion: The inventors have demonstrated that it is possible to express the promiscuous self-TCR of the invention on the surface of human Tregs.

### Example 6: Promiscuous Self-TCR Tregs Bind to Peptide-HLA Dextramers

Method: To demonstrate that the promiscuous self-TCR on Tregs can bind to the Sm autoantigen, the Ro autoantigen and the MPO autoantigen, the inventors generated the following peptide-HLA dextramers—SmD1:78-21/HLA-DRB1*03:01, Ro60:369-383/HLA-DRB1*03:01 and MPO:453-567/HLA-DRB1*04:05, then measured binding by flow cytometry.

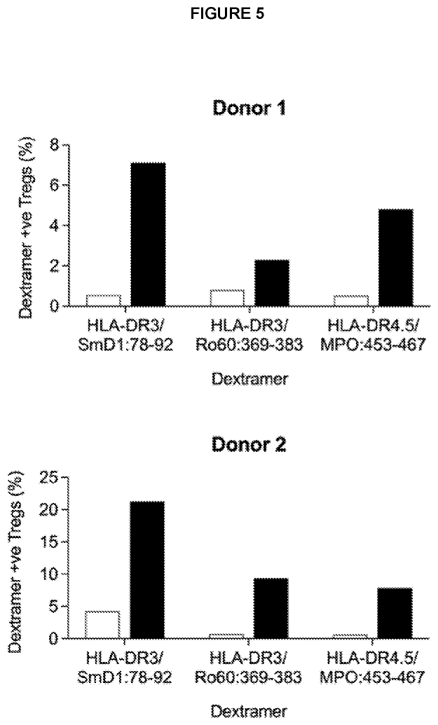

Result: As shown in FIG. 5, Tregs transduced with the promiscuous self-TCR exhibit binding to the SmD1:78-21/HLA-DRB1*03:01, Ro60:369-383/HLA-DRB1*03:01 and MPO:453-567/HLA-DRB1*04:05 dextramers.

Conclusion: Tregs transduced with the promiscuous self-TCR are able to bind promiscuously to the Sm autoantigen, Ro60 autoantigen and MPO autoantigen.

### Example 7: Promiscuous Self-TCR Tregs are Activated Following Peptide Stimulation

Method: To demonstrate that promiscuous self-TCR Tregs are activated upon peptide stimulation, promiscuous self-TCR transduced Tregs were co-cultured with HLA-DRB1*03:01/HLA-DRB1*04:05 expressing B-LCLs and either SmD1:78-92,Ro60:369-383, or MPO:453-467, then the early T cell activation marker CD69 and the Treg-specific activation marker GARP were measured by flow cytometry. The upregulation of CD69 or GARP was compared to mock transduced Tregs.

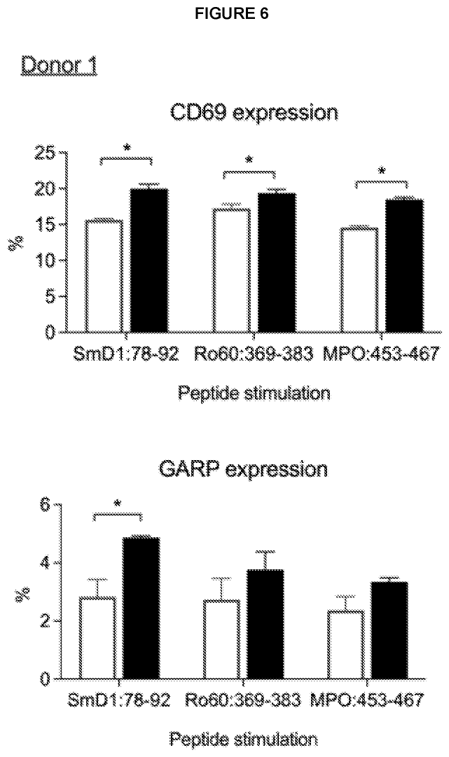

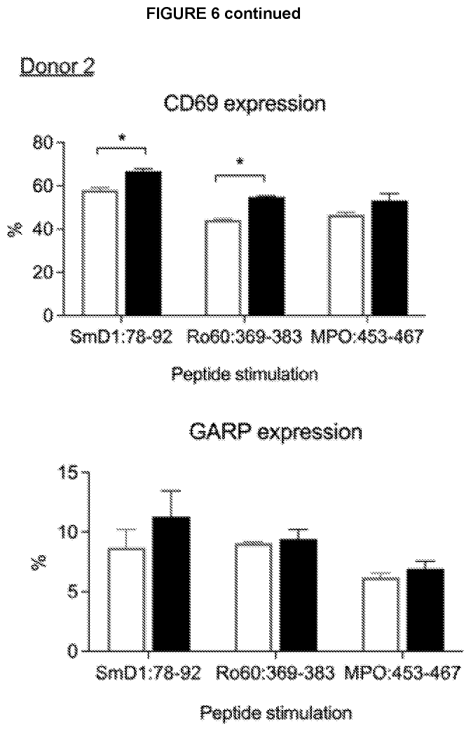

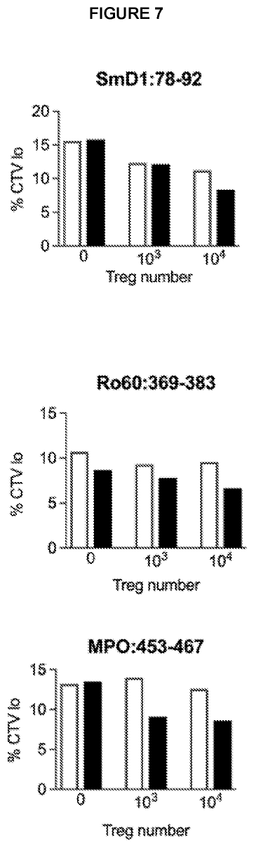

Result: As shown in FIG. 6, stimulation with SmD1:78-92, Ro60:369-383 and MPO:453-467 induced activation of promiscuous self-TCR transduced Tregs compared to no peptide stimulation.

Conclusion: Promiscuous self-TCR transduced Tregs are activated upon stimulation with either the Sm autoantigen, Ro60 autoantigen and the MPO autoantigen.

### Example 8: Promiscuous Self-TCR Engages Sm Peptides

Method: To demonstrate that the promiscuous self-TCR can engage Sm-peptide HLA-complexes, the inventors used imaging flow cytometry to visualise the immune synapse between promiscuous self-TCR expressing J76 T cells and HLA-DRB1*03:01 expressing B-LCLs as antigen presenting cells. BLCLs were pulsed with either no peptide, SmD1:78-92 or SmB/B′:7-21 for 2 hours in serum free RPMI at 37° C. Pulsed B-LCLs were then mixed with the promiscuous self-TCR transduced J76 cells at a 1:1 ratio in serum free RPMI and incubated for 2 hours at 37° C. Cells were then fixed and stained with an antibody/phalloidin cocktail containing anti-HLA-DR BV421 (clone G46-6, BD), anti-CD3e PE (clone OKT3, Invitrogen) and phalloidin AF647 (Invitrogen) in permeabilization buffer. Cells were resuspended in PBS and propidium iodide added just prior to acquisition on an Amnis Imagestream X Mark II imaging flow cytometer (Luminex) followed by analysis using Ideas Software ver.6.2 (Luminex) to identify and quantify the fluorescence of the immune synapse of the B-LCL-J76 doublets. An increase in mean pixel intensity of either CD3 or phalloidin at the immune synapse signals TCR binding and activation.

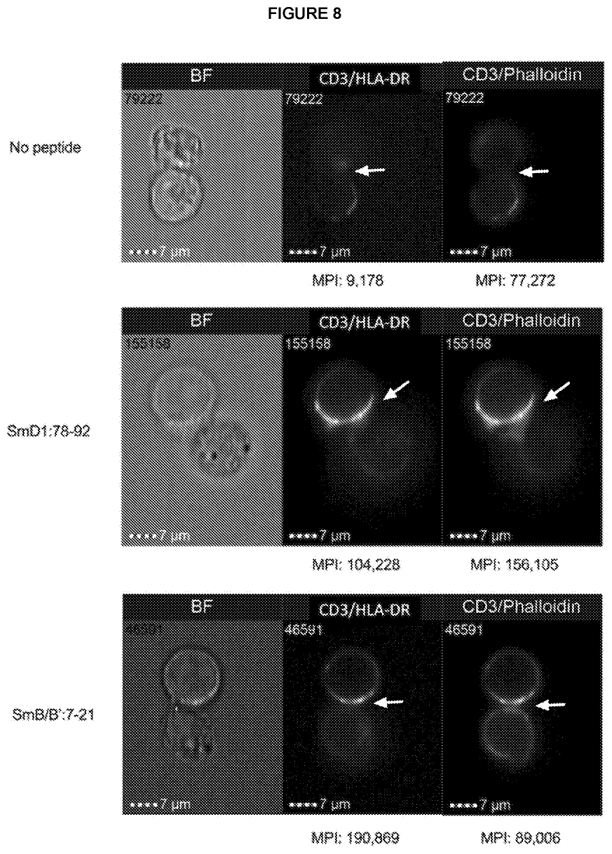

Result: As shown in FIG. 8, CD3 and phalloidin mean pixel intensity at the immune synapse was increased when B-LCLs were pulsed with the Sm epitopes.

Conclusion: The promiscuous self-TCR can engage both HLA-DRB1*03:01 restricted Sm epitopes.

It will be understood that the invention disclosed and defined in this specification extends to all alternative combinations of two or more of the individual features mentioned or evident from the text or drawings. All of these different combinations constitute various alternative aspects of the invention.

1. A method of treating an autoimmune or inflammatory disease, the method comprising:
administering to a subject in need thereof, a population of regulatory T (T reg) cells expressing a binding protein on their surface,
wherein the binding protein comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
wherein the Vβ domain comprises a CDR3 having an amino acid sequence as set forth in SEQ ID NO: 16, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto;
thereby treating the autoimmune disease or inflammatory disease,
wherein the autoimmune or inflammatory disease is characterised by an aberrant or inappropriate immune response to:
Ro60 protein;
MPO protein;
Ro60 protein and MPO protein;
Smith protein and Ro60 protein;
Smith protein and MPO protein; or
Smith protein, Ro60 protein and MPO protein.

2. The method of claim 1, wherein the autoimmune or inflammatory disease is characterised by an aberrant or inappropriate immune response to Smith protein.

3. The method of claim 1, wherein the autoimmune or inflammatory disease is characterised by an aberrant or inappropriate immune response to Ro60 protein.

4. The method of claim 1, wherein the autoimmune or inflammatory disease is characterised by an aberrant or inappropriate immune response to Smith protein and to Ro60 protein.

5. The method of claim 1, wherein the autoimmune or inflammatory disease is characterised by an aberrant or inappropriate immune response to MPO protein.

6. The method of claim 1, wherein the autoimmune or inflammatory disease is characterised by an aberrant or inappropriate immune response to Ro60 protein and to MPO protein.

7. The method of claim 1, wherein the autoimmune or inflammatory disease is characterised by an aberrant or inappropriate immune response to Smith protein, to Ro60 protein and to MPO protein.

8. The method of any one of claims 1 to 7, wherein the aberrant or inappropriate immune response to Smith protein, to Ro60 protein and/or to MPO protein, comprises the formation of auto-antibodies to Smith protein, to Ro60 protein and/or to MPO protein.

9. The method of any one of claims 1 to 8, wherein the autoimmune or inflammatory disease is one or more selected from the group consisting of: systemic lupus erythematosus (SLE), lupus nephritis, Sjogren's Syndrome, systemic sclerosis, inflammatory myositis, inflammatory rheumatism, autoimmune vasculitis, and microscopic polyangiitis.

10. The method of claim 9, wherein the autoimmune or inflammatory disease is SLE or lupus nephritis.

11. The method of claim 9, wherein the autoimmune or inflammatory disease is Sjogren's Syndrome, preferably primary Sjogren's Syndrome.

12. The method of claim 9, wherein the autoimmune or inflammatory disease is vasculitis, preferably microscopic polyangiitis (MPA).

13. The method of claim 9, wherein the subject requires treatment for SLE and Sjogren's Syndrome, or for SLE and MPA, or for Sjogren's Syndrome and MPA, or for all three of SLE, Sjogren's Syndrome and MPA.

14. A method of treating systemic lupus erythematosus (SLE) or lupus nephritis in a subject, the method comprising:
administering to a subject in need thereof, a population of T reg cells expressing a binding protein on their surface,
wherein the binding protein comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
wherein the Vβ domain comprises a CDR3 having an amino acid sequence as set forth in SEQ ID NO: 16, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto;
thereby treating the SLE or lupus nephritis in the subject,
wherein the SLE or lupus nephritis is characterised by an aberrant or inappropriate immune response to:
Ro60 protein; or
Smith protein and Ro60 protein.

15. A method of treating Sjogren's Syndrome in a subject, the method comprising administering to a subject in need thereof, a population of T reg cells expressing a binding protein on their surface,
wherein the binding protein comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
wherein the Vβ domain comprises a CDR3 having an amino acid sequence as set forth in SEQ ID NO: 16, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto;
thereby treating the Sjogren's Syndrome in the subject.

16. A method of treating autoimmune vasculitis, preferably microscopic polyangiitis (MPA) in a subject, the method comprising:
administering to a subject in need thereof, a population of T reg cells expressing a binding protein on their surface,
wherein the binding protein comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
wherein the Vβ domain comprises a CDR3 having an amino acid sequence as set forth in SEQ ID NO: 16, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto;
thereby treating the vasculitis, preferably microscopic polyangiitis in the subject.

17. The method of claim 14, wherein the method also comprises treating Sjogren's Syndrome and/or MPA in the subject.

18. The method of claim 15, wherein the method also comprises treating SLE, lupus nephritis and/or MPA in the subject.

19. The method of claim 16, wherein the method also comprises treating SLE, lupus nephritis and/or Sjogren's Syndrome in the subject. 20 The method of any one of claims 15 to 19, wherein the Sjogren's Syndrome or MPA is characterised by an aberrant immune response to one or more of Smith protein, Ro60 protein and MPO protein in the subject.

21. The method of claim 20, wherein the aberrant immune response comprises the formation of autoantibodies to one or more of the Smith protein, Ro60 protein and MPO protein in the subject.

22. The method of any one of the preceding claims, wherein the binding protein comprises:
a Vα domain comprising a CDR1 having an amino acid sequence as set forth in SEQ ID NO: 11, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
a Vα domain comprising a CDR2 having an amino acid sequence as set forth in SEQ ID NO: 12 or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
a Vβ domain comprising a CDR1 having an amino acid sequence as set forth in SEQ ID NO: 14, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
a Vβ domain comprising a CDR2 having an amino acid sequence as set forth in SEQ ID NO: 15, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto.

23. The method of any one of the preceding claims, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2 and a CDR3 comprising the amino acid sequences set forth in SEQ ID NOs: 14, 15 and 16.

24. The method of claim 1, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16.

25. The method of claim 2, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16.

26. The method of claim 3, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16.

27. The method of claim 4, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16.

28. The method of claim 5, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16.

29. The method of claim 6, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16.

30. The method of claim 7, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16.

31. The method of claim 8, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16 and wherein the aberrant or inappropriate immune response to Smith protein, to Ro60 protein and to MPO protein, comprises the formation of auto-antibodies to Smith protein, to Ro60 protein and to MPO protein.

32. The method of claim 14, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2 and a CDR3 comprising the amino acid sequences set forth in SEQ ID NOs: 14, 15 and 16.

33. The method of claim 15, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16.

34. The method of claim 16, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16.

35. The method of claim 15, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16 and the Sjogren's Syndrome is characterised by an aberrant immune response to one or more of Smith protein, Ro60 protein and MPO protein in the subject.

36. The method of claim 16, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2and a CDR3 comprising the amino acid sequences set forth in SEQ ID NOs: 14, 15 and 16 and wherein the MPA is characterised by an aberrant immune response to one or more of Smith protein, Ro60 protein and MPO protein in the subject

37. The method of any one of the preceding claims, wherein the binding protein comprises a TCRα chain comprising or consisting of an amino acid sequence as set forth in SEQ ID NO: 23 with 1-10 amino acid insertions, deletions, substitutions, additions or a combination thereof, outside the indicated CDR sequences.

38. The method of any one of the preceding claims, wherein the binding protein comprises a TCRβ chain comprising or consisting of an amino acid sequence as set forth in SEQ ID NO: 24 with 1-10 amino acid insertions, deletions, substitutions, additions or a combination thereof, outside the indicated CDR sequences.

39. The method of any one of the preceding claims, wherein the binding protein comprises a TCRα chain comprising or consisting of an amino acid sequence as set forth in SEQ ID NO: 23, and a TCRβ chain comprising or consisting of an amino acid sequence as set forth in SEQ ID NO: 24.

40. The method of any one of the preceding claims, wherein the binding protein is capable of binding to a complex of a fragment of a Smith protein, Ro60 protein or MPO protein, and an HLA-DR3 molecule or HLA-DR4 molecule.

41. The method of any one of the preceding claims, wherein the binding protein is capable of binding to a complex of a fragment of a Smith protein and an HLA-DR3 molecule, wherein the fragment of the Smith protein comprises or consists of an amino acid sequence of, or equivalent to residues 78-92 of an SmD1 protein or residues 7-21 of a SmB/B′ protein (as set forth in SEQ ID NOs: 1 and 3, respectively).

42. The method of any one of the preceding claims, wherein the binding protein is capable of binding to a complex of a fragment of Ro60 protein and an HLA-DR3 molecule, wherein the fragment of the Ro60 protein comprises or consists of an amino acid sequence of, or equivalent to residues 225-239 or residues 369-383 of Ro60, as set forth in SEQ ID NOs: 5 and 6, respectively.

43. The method of any one of the preceding claims, wherein the binding protein is capable of binding to a complex of a fragment of MPO protein and an HLA-DR4 molecule, wherein the fragment of the MPO protein comprises or consists of an amino acid sequence of, or equivalent to residues 453-467 or residues 724-738 of MPO, as set forth in SEQ ID NOs: 8 and 9, respectively.

44. The method of any one of the preceding claims, wherein the TCRα chain and TCRβ chain are modified to include a cysteine residue that allows formation of an additional interchain disulfide bond.

45. The method of claim 44, wherein the residue at, or equivalent to, Thr48 on the TCRα chain and the residue at, or equivalent to, Ser57 on the TCRβ chain are replaced with cysteines to facilitate the creation of the additional disulfide bond between the TCR constant regions.

46. The method of any one of the preceding claims, wherein the population of T reg cells is derived from the subject requiring treatment.

47. The method of any one of claims 1 to 46, wherein the population of T reg cells is derived from stem cells, optionally wherein the stem cells are induced pluripotent stem cells (iPSCs) or embryonic stem cells.

48. The method of any one of claims 1 to 46, wherein the population of T reg cells is derived from a mixed population of T cells in which a nucleic acid encoding a binding protein as defined in any one of claims 1 to 46 was introduced.

49. Use of a population of regulatory T (T reg) cells expressing a binding protein on their surface, in the manufacture of a medicament for treating an autoimmune disease or inflammatory disease in a subject,
wherein the binding protein on the surface of the T reg cells comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
wherein the Vβ domain comprises a CDR3 having an amino acid sequence as set forth in SEQ ID NO: 16, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto;
wherein the autoimmune or inflammatory disease is characterised by an aberrant or inappropriate immune response to:
Ro60 protein;
MPO protein;
Ro60 protein and MPO protein;
Smith protein and Ro60 protein;
Smith protein and MPO protein; or
Smith protein, Ro60 protein and MPO protein.

50. The use of claim 49, wherein the autoimmune or inflammatory disease is characterised by an aberrant or inappropriate immune response to Smith protein.

51. The use of claim 49, wherein the autoimmune or inflammatory disease is characterised by an aberrant or inappropriate immune response to Ro60 protein.

52. The use of claim 49, wherein the autoimmune or inflammatory disease is characterised by an aberrant or inappropriate immune response to Smith protein and to Ro60 protein.

53. The use of claim 49, wherein the autoimmune or inflammatory disease is characterised by an aberrant or inappropriate immune response to MPO protein.

54. The use of claim 49, wherein the autoimmune or inflammatory disease is characterised by an aberrant or inappropriate immune response to Ro60 protein and to MPO protein.

55. The use of claim 49, wherein the autoimmune or inflammatory disease is characterised by an aberrant or inappropriate immune response to Smith protein, to Ro60 protein and to MPO protein.

56. The use of any one of claims 49 to 55, wherein the aberrant or inappropriate immune response to Smith protein, to Ro60 protein and/or to MPO protein, comprises the formation of auto-antibodies to Smith protein, to Ro60 protein and/or to MPO protein.

57. The use of any one of claims 49 to 56, wherein the autoimmune or inflammatory disease is one or more selected from the group consisting of: systemic lupus erythematosus (SLE), lupus nephritis, Sjogren's Syndrome, systemic sclerosis, inflammatory myositis, inflammatory rheumatism, autoimmune vasculitis, and microscopic polyangiitis.

58. The use of claim 57, wherein the autoimmune or inflammatory disease is SLE or lupus nephritis.

59. The use of claim 57, wherein the autoimmune or inflammatory disease is Sjogren's Syndrome, preferably primary Sjogren's Syndrome.

60. The use of claim 57, wherein the autoimmune or inflammatory disease is vasculitis, preferably microscopic polyangiitis (MPA).

61. The use of claim 57, wherein the subject requires treatment for SLE and Sjogren's Syndrome, or for SLE and MPA, or for Sjogren's Syndrome and MPA, or for all three of SLE, Sjogren's Syndrome and MPA.

62. Use of a population of T reg cells expressing a binding protein on their surface, in the manufacture of a medicament for treating systemic lupus erythematosus (SLE) or lupus nephritis in a subject,
wherein the binding protein on the surface of the T reg cells comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
wherein the Vβ domain comprises a CDR3 having an amino acid sequence as set forth in SEQ ID NO: 16, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto;
wherein the SLE or lupus nephritis is characterised by an aberrant or inappropriate immune response to:
Ro60 protein; or
Smith protein and Ro60 protein.

63. Use of a population of T reg cells expressing a binding protein on their surface, in the manufacture of a medicament for treating Sjogren's Syndrome in a subject,
wherein the binding protein on the surface of the T cells comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
wherein the Vβ domain comprises a CDR3 having an amino acid sequence as set forth in SEQ ID NO: 16, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto.

64. Use of a population of T reg cells expressing a binding protein on their surface, in the manufacture of a medicament for treating autoimmune vasculitis, preferably microscopic polyangiitis (MPA) in a subject,
wherein the binding protein on the surface of the T reg cells comprises a T cell receptor (TCR) α-chain variable domain (Vα) and a TCR β-chain variable domain (Vβ),
wherein the Vα domain comprises a CDR3 having an amino acid sequence as set forth in SEQ ID NO: 13, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
wherein the Vβ domain comprises a CDR3 having an amino acid sequence as set forth in SEQ ID NO: 16, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto.

65. The use of claim 62, wherein the medicament is also for treating Sjogren's Syndrome and/or MPA in the subject.

66. The use of claim 63, wherein the medicament is also for treating SLE, lupus nephritis and/or MPA in the subject.

67. The use of claim 64, wherein the medicament is also for treating SLE, lupus nephritis and/or Sjogren's Syndrome in the subject.

68. The use of any one of claims 63 to 67, wherein the Sjogren's Syndrome or MPA is characterised by an aberrant immune response to one or more of Smith protein, Ro60 protein and MPO protein in the subject.

69. The use of claim 68, wherein the aberrant immune response comprises the formation of autoantibodies to one or more of the Smith protein, Ro60 protein and MPO protein in the subject.

70. The use of any one of claims 49 to 69, wherein the binding protein comprises:
a Vα domain comprising a CDR1 having an amino acid sequence as set forth in SEQ ID NO: 11, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
a Vα domain comprising a CDR2 having an amino acid sequence as set forth in SEQ ID NO: 12 or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
a Vβ domain comprising a CDR1 having an amino acid sequence as set forth in SEQ ID NO: 14, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto; and
a Vβ domain comprising a CDR2 having an amino acid sequence as set forth in SEQ ID NO: 15, or a sequence at least about 80%, at least about 85%, at least about 90%, at least about 91%, at least about 92%, at least about 93%, at least about 94%, at least about 95%, at least about 96%, at least 97%, at least about 98%, or at least about 99% identical thereto.

71. The use of any one of claims 49 to 70, wherein the binding protein comprises a Vα domain having a CDR1, a CDR2 and a CDR3 comprising the amino acid sequences set forth in SEQ ID NOs: 11, 12 and 13 and a Vβ domain having a CDR1, a CDR2 and a CDR3 comprising the amino acid sequences set forth in SEQ ID NOs: 14, 15 and 16.

72. The use of any one of claims 49 to 71, wherein the binding protein comprises a TCRα chain comprising or consisting of an amino acid sequence as set forth in SEQ ID NO: 23 with 1-10 amino acid insertions, deletions, substitutions, additions or a combination thereof, outside the indicated CDR sequences.

73. The use of any one of claims 49 to 72, wherein the binding protein comprises a TCRβ chain comprising or consisting of an amino acid sequence as set forth in SEQ ID NO: 24 with 1-10 amino acid insertions, deletions, substitutions, additions or a combination thereof, outside the indicated CDR sequences.

74. The use of any one of claims 49 to 73, wherein the binding protein comprises a TCRα chain comprising or consisting of an amino acid sequence as set forth in SEQ ID NO: 23, and a TCRβ chain comprising or consisting of an amino acid sequence as set forth in SEQ ID NO: 24.

75. The use of any one of claims 49 to 74, wherein the binding protein is capable of binding to a complex of a fragment of a Smith protein, Ro60 protein or MPO protein, and an HLA-DR3 molecule.

76. The use of any one of claims 49 to 75, wherein the binding protein is capable of binding to a complex of a fragment of a Smith protein and an HLA-DR3 molecule, wherein the fragment of the Smith protein comprises or consists of an amino acid sequence of, or equivalent to residues 78-92 of an SmD1 protein or residues 7-21of a SmB/B′ protein (as set forth in SEQ ID NOs: 1 and 3, respectively).

77. The use of any one of claims 49 to 76, wherein the binding protein is capable of binding to a complex of a fragment of Ro60 protein and an HLA-DR3 molecule, wherein the fragment of the Ro60 protein comprises or consists of an amino acid sequence of, or equivalent to residues 225-239 or residues 369-383 of Ro60, as set forth in SEQ ID NOs: 5 and 6, respectively.

78. The use of any one of claims 49 to 77, wherein the binding protein is capable of binding to a complex of a fragment of MPO protein and an HLA-DR4 molecule, wherein the fragment of the MPO protein comprises or consists of an amino acid sequence of, or equivalent to residues 453-467 or residues 724-738 of MPO, as set forth in SEQ ID NOs: 8 and 9, respectively.

79. The use of any one of claims 49 to 78, wherein the population of T reg cells is derived from the subject requiring treatment.

80. The use of any one of claims 49 to 78, wherein the population of T reg cells is derived from stem cells, optionally wherein the stem cells are induced pluripotent stem cells (iPSCs) or embryonic stem cells.

81. The use of any one of claims 49 to 79, wherein the population of T reg cells is derived from a mixed population of T cells in which a nucleic acid encoding a binding protein as defined in any one of claims 1 to 45 was introduced.
